(19) United States
(12) Patent Application Publication
(10) Pub. No.: US 2025/0375220 A1
(43) Pub. Date: Dec. 11, 2025

(54) FRICTION-FIT IMPLANTABLE DEVICES AND ASSEMBLIES

(71) Applicant: Orthofix US LLC, Lewisville, TX (US)

(72) Inventors: Corey Gladieux, Vista, CA (US); Michael Krasovic, San Diego, CA (US); Geoffrey Toon, San Diego, CA (US); Kelsey Witt, Carlsbad, CA (US)

(21) Appl. No.: 19/227,142

(22) Filed: Jun. 3, 2025

Related U.S. Application Data
(60) Provisional application No. 63/656,949, filed on Jun. 6, 2024.

(51) Int. Cl. A61B 17/70

(52) U.S. Cl. CPC A61B 17/7032; A61B 17/7035

(57) ABSTRACT

Implantable devices and assemblies that provide a friction fit between a receiver and a fastener, such as a bone screw. A frictional force is applied between the screw head and both a retention ring and a pressure insert, maintaining and stabilizing the orientation of the receiver relative to the screw head before being fixed by a set screw. This mechanism prevents drooping or slipping of the receiver out of alignment when implanted. Additionally, the devices allow for modular assembly before or during spinal fixation, enabling bottom-side loading of the screw head into the receiver. Various screws with different characteristics can be coupled to the receiver body before or after implantation. The frictional force may be facilitated by one or more ridges on the receiver body that engage the pressure insert, which is resiliently urged against the screw head, providing a spring-like compression force.

### PRIORITY

This application claims the benefit of the filing date of U.S. Provisional Application 63/656,949, filed Jun. 6, 2024, which is incorporated herein in its entirety.

### TECHNICAL FIELD

This disclosure is generally directed to friction-fit devices and assemblies attachable to bone fasteners for implantation in an anatomy of a patient. For instance, one or more implantable assemblies including a receiver body coupled to a bone screw can be coupled to a connecting rod to retain one or more vertebrae in a desired relationship.

### BACKGROUND

Various systems for connecting fasteners (e.g., pedicle screws) to elongated supports (e.g., fixation rods) for the purposes of vertebral fixation have been proposed. Although described with reference to vertebral or spinal fixation, it should be appreciated that the systems described herein can be similarly applicable to other bone structures as well.

Generally, fixation systems include a receiver (or “receiver body” or “receiver head”) which is attachable to both a fastener and a fixation rod to retain the rod in fixed relation to the fastener, and in turn, a vertebra into which the fastener is secured. Traditional receiver assemblies include a receiver and a fastener for attachment of one or more fixation rods to a vertebra. A physician can use multiple receiver assemblies and/or multiple rods to secure the vertebrae in a desired spatial relationship. In some installations, a first rod can extend along a first side of a patient's spine and engage a first plurality of fastener assemblies each implanted in a different vertebra, and a second rod can extend along a second side of the patient's spine and engage a second plurality of fastener assemblies.

In some instances, a receiver assembly can be preassembled such that the receiver and fastener are preselected and attached to one another by the manufacturer. The assembly of the fastener and the receiver can involve special tools and trained technicians such that assembly by the physician, nurse, or surgical technician is impractical. Accordingly, the surgeon or technician can select a receiver and fastener assembly from a plurality of receiver and fastener assemblies based on the patient's anatomy and/or indications. Thus, the surgeon can be limited based on the variety of selections available at the time of surgery.

During a spinal fixation surgery, the receiver and fastener assemblies can be inserted through the patient's tissue via a surgical opening or ingress. The fasteners of each assembly can be driven into the patient's vertebra at desired locations. A connecting rod is then positioned through each receiver and the receivers and connecting rod are fixed in place by set screws or compression screws in each receiver. In order to position the connecting rod through each receiver, the receivers are oriented in alignment so that the connecting rod can be inserted through a channel or slot of each receiver. The alignment of the receivers can be a complicated part of the procedure. For example, gravity can cause the receivers to droop or slip out of alignment. Accordingly, the procedure can involve repositioning and/or reorienting one or more receivers multiple times before the connecting rod is successfully positioned through each receiver.

### SUMMARY

The present disclosure describes implantable devices and assemblies that provide a friction fit between a receiver and a fastener (e.g., bone screw). For example, a frictional force can be applied between a screw head and a retention ring, and another frictional force can be applied between the screw head and a pressure insert. The frictional force and contact maintains and stabilizes an orientation of a receiver relative to the screw head before the position is fixed by a set screw. This friction fit mechanism reduces or prevents drooping or slipping of the receiver out of alignment when the fastener is implanted into the bone and prior to locking with a set screw. Further, the implantable devices of the present disclosure can allow for modular assembly before or during a spinal fixation procedure. For example, the implantable device can allow for bottom-side loading of the screw head up into the receiver so that various screws having various characteristics (e.g., length, diameter, etc.) can be coupled to the receiver body before or after the bone screw has been implanted into bone. The frictional force can be facilitated by one or more ridges or protrusions on an interior surface of the receiver body that engage a rounded surface or edge of the pressure insert. The engagement can be such that the pressure insert is resiliently urged against the head of the screw. The depression or groove of the pressure insert can be disposed on one or more wings, arms, or tabs of the pressure insert. The one or more wings, arms, or tabs can be at least partially resilient or compliant to allow for a spring-like compression force between the pressure insert and the head of the bone screw.

In an exemplary implementation, the present disclosure is directed to a fastener assembly for a spinal fixation system. The fastener assembly can include a bone shank comprising a head portion and a distal threaded portion configured to be implanted into bone. The fastener assembly can also include a receiver comprising a channel for receiving a fixation rod therein; an axial bore extending longitudinally through the receiver from a proximal opening of the receiver to a distal opening, the distal opening being sized and shaped to receive the head portion of the bone shank therethrough; a chamber disposed adjacent to the distal opening, the chamber comprising a conical interior surface, the conical interior surface comprising a distal portion having a first diameter, and a proximal portion having a second diameter larger than the first diameter; and a ridge protruding from a surface of the axial bore. A split retainer ring can be disposed in the chamber. The split retainer ring can be configured to expand within the chamber to allow the head portion of the bone shank to pass therethrough and thereafter support the head portion in a pivotable relationship. A pressure insert can be disposed at least partly above the split retainer ring. The pressure insert can include a saddle configured to seat the fixation rod, a distally-facing concave surface configured to contact the head portion of the bone shank, and a depression formed in an exterior surface of the pressure insert. The pressure insert also can include a rounded corner adjacent to a proximal end of the pressure insert. When the pressure insert is in a first longitudinal position relative to the receiver, the depression can be configured to engage the ridge in the receiver to maintain the pressure insert at the first longitudinal position. When the pressure insert is in a second longitudinal position distal, the ridge can be disengaged from the depression and urged against the rounded corner of the pressure insert such that the head portion of the bone shank is maintained in a friction fit with both the pressure insert and the split retainer ring.

In some aspects, the head portion of the bone shank comprises a spherical surface, wherein the split retainer ring comprises an interior concave surface configured to contact the spherical surface to thereby provide the pivotable relationship. In some aspects, the receiver comprises an interior conical surface defining the chamber, the split retainer ring further comprises an outer conical surface, and when the pressure insert is in the second longitudinal position, the outer conical surface abuts the interior conical surface of the receiver. In some aspects, the split retainer ring comprises a groove formed in a proximal end of the split retainer ring, wherein a distal end of the pressure insert is seated in the groove when the pressure insert is in the second longitudinal position. In some aspects, the fastener assembly can include a compression screw configured to be threadably received into an upper opening of the receiver along a longitudinal axis of the receiver, and wherein the compression screw is configured to compress the fixation rod against the pressure insert, which in turn locks the fastener assembly by simultaneously urging: the spherical surface of the head portion of the bone shank against the interior concave surface of the split retainer ring; and the outer conical surface of the split retainer ring against the interior conical surface of the receiver. In some aspects, the depression comprises a groove extending circumferentially across the exterior surface of the pressure insert. In some aspects, the groove comprises a non-symmetrical cross-sectional profile. In some aspects, the ridge comprises a symmetrical cross-sectional profile.

In another exemplary aspect, the present disclosure is directed to a receiver for a polyaxial bone screw assembly, and the receiver may include a body. The body may include a rod receiving channel sized and shaped to receive a fixation rod therein and an axial bore extending longitudinally through the receiver to a distal opening. The distal opening can be sized and shaped to receive a head of a bone shank therethrough. A conical chamber can be disposed adjacent to the distal opening. The conical chamber can be wider at a proximal end of the conical chamber than at a distal end of the conical chamber. A ridge can protrude from a surface of the axial bore. A split retainer ring can be disposed in the conical chamber. The split retainer ring can include a conical exterior surface, wherein the split retainer ring is configured to expand within the conical chamber to allow a head portion of a bone shank to pass therethrough and thereafter support the head portion in a pivotable relationship. A pressure insert can be disposed at least partly above the split retainer ring. The pressure insert can include a saddle surface configured to seat the fixation rod; a distally-facing concave surface configured to abut the head portion of the bone shank; a groove formed in an exterior surface of the pressure insert; and a rounded corner adjacent to a proximal end of the pressure insert. In some aspects, the groove is configured to engage the ridge in the receiver to maintain the pressure insert in a first state associated with a first longitudinal position within the receiver. In addition, the pressure insert can be configured to flex inward to disengage the groove from the ridge, and the groove can be configured to abut the rounded corner of the pressure insert in a second state associated with a second longitudinal position within the receiver. In the second state, the groove can urge the pressure insert distally such that: the head portion of the bone shank forms a first friction fit with the concave surface of the pressure insert; the head portion of the bone shank forms a second friction fit with an interior surface of the split retainer ring; and the conical exterior surface of the split retainer ring is urged against a surface of the conical chamber.

In an aspect, the split retainer ring comprises an interior concave surface. In an aspect, the interior concave surface comprises a spherical surface. In an aspect, the receiver comprises an interior conical surface defining the chamber, and the split retainer ring further comprises an outer conical surface abutting the interior conical surface of the receiver. In an aspect, the split retainer ring comprises a groove formed in a proximal end of the split retainer ring, and wherein a distal end of the pressure insert is positioned adjacent the groove when the pressure insert is in the second longitudinal position. In an aspect, the groove extends circumferentially across the exterior surface of the pressure insert. In an aspect, the groove comprises a non-symmetrical cross-sectional profile. In an aspect, the ridge comprises a symmetrical cross-sectional profile.

In another exemplary aspect, the present disclosure is directed to a receiver body for a polyaxial bone screw assembly. The receiver body may include a longitudinal axis and a rod receiving channel oriented transverse to the longitudinal axis and being sized and shaped to receive a fixation rod therein. An axial bore can extend along the longitudinal axis from a proximal opening to a distal opening. The distal opening can be sized and shaped to receive a head of a bone shank therethrough. A conical chamber can be disposed adjacent to the distal opening and in communication with the axial bore, the conical chamber being wider at a proximal end of the conical chamber than at a distal end of the conical chamber. The conical chamber can be wider than the axial bore at both the proximal end and the distal end of the conical chamber. A ridge can protrude inward into the axial bore from an interior surface surrounding the axial bore, the ridge can be disposed proximally of the conical chamber. A set of threads can be on a proximal portion of the interior surface and disposed proximally of the ridge.

In an aspect, the receiver body can include a ledge disposed between the conical chamber and the distal opening. In an aspect, the ridge comprises a symmetrical cross-sectional profile. In an aspect, the symmetrical cross-sectional profile comprises a circular arc, a gaussian curve, or an elliptical arc.

In another exemplary aspect, the present disclosure is directed to a method for assembling a polyaxial fastener assembly. The method can include inserting a head portion of a bone shank through a distal opening of a receiver assembly and into a chamber of the receiver assembly. The receiver assembly can include a receiver body, comprising: the distal opening; a conical interior surface disposed about the chamber; an axial bore extending longitudinally through the receiver; and a ridge protruding from a surface of the axial bore. The receiver assembly can also include a split retainer ring disposed in the chamber and a pressure insert disposed at least partially within the axial bore. The pressure insert can include a distally-facing concave surface configured to contact the head portion of the bone shank; a groove formed in an exterior surface of the pressure insert; and a rounded corner adjacent to a proximal end of the pressure insert. The inserting step can include pushing the head portion through the distal opening of the receiver body and through the split retainer ring to cause the split retainer ring to elastically expand about the head portion, and thereafter to collapse about a neck of the bone shank to retain the head portion within the chamber in a pivotable relationship with the receiver body. The method also may include urging the pressure insert from a first longitudinal position to a second longitudinal position to create a friction fit engagement of the head portion with both the concave surface of the pressure insert and the split retainer ring. When the pressure insert is in the first longitudinal position relative to the receiver body, the groove can be engaged with the ridge in the receiver body to maintain the pressure insert at the first longitudinal position. When the pressure insert is in the second longitudinal position, the ridge can be disengaged from the groove and urged against the rounded corner of the pressure insert.

In another exemplary aspect, the present disclosure is directed to method for assembling a polyaxial fastener assembly. The method may include providing a receiver body including a rod receiving channel sized and shaped to receive a fixation rod therein; an axial bore extending longitudinally through the receiver to a distal opening, the distal opening being sized and shaped to receive a head of a bone shank therethrough; a conical chamber disposed adjacent to the distal opening, the conical chamber being wider at a proximal end of the conical chamber than at a distal end of the conical chamber; and a ridge protruding from a surface of the axial bore. The method may include inserting a pressure insert into the receiver body through one of the proximal opening or the distal opening, wherein the step of inserting the pressure insert comprises engaging a depression of the pressure insert with the ridge of the receiver. The method also can include inserting a split retainer ring through the distal opening such that a conical outer surface of the split retainer ring rests against a conical surface of the conical chamber. The step of inserting the split retainer ring comprises compressing the split retainer ring from a first width to a second width, wherein the second width of the split retainer ring is smaller than the width of the distal opening.

In another exemplary aspect, the present disclosure is directed to a fastener kit for a spinal fixation system, the fastener kit may include a bone shank comprising a head portion and a distal threaded portion configured to be implanted into bone. The fastener kit also may include a receiver comprising: a channel configured to receive a fixation rod therein and an axial bore extending longitudinally through the receiver from a proximal opening of the receiver to a distal opening. The distal opening can be sized and shaped to receive the head portion of the bone shank therethrough. A chamber can be disposed adjacent to the distal opening. The chamber can include a conical interior surface comprising a distal portion having a first diameter, and a proximal portion having a second diameter larger than the first diameter. A ridge can protrude from a surface of the axial bore. A split retainer ring can be sized and shaped to be positioned in the chamber, and a pressure insert can be sized and shaped to be positioned at least partly above the split retainer ring. The pressure insert can include a saddle configured to seat the fixation rod, a distally-facing concave surface configured to contact the head portion of the bone shank, a depression formed in an exterior surface of the pressure insert; and a rounded corner adjacent to a proximal end of the pressure insert.

In another exemplary aspect, the present disclosure is directed to a method of assembling a fixation assembly. The method may include engaging a receiver with the head of a screw; engaging a receiver with the head of a screw; inserting a distal opening of the receiver over the head of the screw; capturing the screw head in the receiver by pushing the screw head to engage a ring residing in the distal portion of the receiver; engaging an inserter tool with the proximal end of the receiver; and directing the inserter tool to push the receiver in a distal direction to engage a pressure insert residing within the proximal portion of the receiver. The method also may include forcing the distal portion of the pressure insert to compress the screw head against the proximal portion of the ring; and establishing a friction-fit engagement between the receiver and the screw head.

In some aspects, the method may include inserting a fixation rod into a channel in the receiver; installing a set screw into the proximal portion of the receiver; and securing the set screw against the fixation rod. In some aspects, the method may include inserting the screw into the target vertebra prior to engaging the receiver with the head of the screw.

In yet another exemplary aspect, this disclosure is directed to a fastener assembly for a spinal fixation system. The fastener assembly may include a bone shank comprising a head portion and a distal threaded portion configured to be implanted into bone. The fastener assembly may also include a receiver having a channel for receiving a fixation rod therein and an axial bore extending longitudinally through the receiver from a proximal opening of the receiver to a distal opening. The distal opening may be sized and shaped to receive the head portion of the bone shank therethrough. A chamber may be disposed adjacent to the distal opening, and may include a conical interior surface. The conical interior surface may include a distal portion having a first diameter and a proximal portion having a second diameter larger than the first diameter. The receiver may include a depression in the surface of the axial bore. The fastener assembly also may include a split retainer ring disposed in the chamber and configured to expand within the chamber to allow the head portion of the bone shank to pass therethrough and thereafter support the head portion in a pivotable relationship. A pressure insert may be disposed at least partly above the split retainer ring. The pressure insert can include a saddle configured to seat the fixation rod, a distally-facing concave surface configured to contact the head portion of the bone shank, and a ridge protruding from the exterior surface of the pressure insert. When the pressure insert is in a first longitudinal position relative to the receiver, the ridge is configured to engage the depression in the receiver to maintain the pressure insert at the first longitudinal position. When the pressure insert is in a second longitudinal position, the ridge is disengaged from the depression and positioned in a manner that the head portion of the bone shank is maintained in a friction fit with both the pressure insert and the split retainer ring.

In some aspects, the receiver comprises a rounded corner adjacent the depression, and when the pressure insert is in the second longitudinal position, the ridge is positioned adjacent the rounded corner to maintain the bone shank in the friction fit with both the pressure insert and the split retainer ring. In some aspects, the head portion of the bone shank comprises a spherical surface, wherein the split retainer ring comprises an interior concave surface configured to contact the spherical surface to thereby provide the pivotable relationship.

These and other objects, features and advantages of this invention will become apparent from the following detailed description of the various aspects and principles of the invention taken in conjunction with the accompanying drawings.

### BRIEF DESCRIPTION OF THE DRAWINGS

The accompanying drawings, which are incorporated in and constitute a part of the specification, illustrate embodiments of the invention and, together with the detailed description herein, serve to explain the principles of the invention. The drawings are only for purposes of illustrating examples and are not to be construed as limiting the invention. It is emphasized that, in accordance with the standard practice in the industry, various features are not drawn to scale. In fact, the dimensions of the various features can be arbitrarily increased or reduced for clarity of discussion.

FIG. 1 is a perspective view of a spinal fixation system including friction-fit pedicle screw assemblies and a connecting rod in accordance with an embodiment of the present disclosure.

FIG. 2 is an exploded view of a friction-fit pedicle screw assembly in FIG. 1 in accordance with an embodiment of the present disclosure.

FIG. 3 is a perspective view of a receiver for a friction-fit pedicle screw assembly in accordance with an embodiment of the present disclosure.

FIG. 4A is a cross-sectional view of a body for a friction-fit pedicle screw assembly in FIG. 3.

FIG. 4B is a cross-sectional view of threading on a body in FIG. 4A.

FIG. 4C is a cross-sectional view of a body in FIG. 3.

FIG. 5A is a perspective view of a pressure cap for a friction-fit pedicle screw assembly in accordance with an embodiment of the present disclosure.

FIG. 5B is a top view of a pressure cap for a friction-fit pedicle screw assembly in accordance with an embodiment of the present disclosure.

FIG. 5C is a cross-sectional view of a pressure cap for a friction-fit pedicle screw assembly in accordance with an embodiment of the present disclosure.

FIG. 6A is a perspective view of a retainer ring for a friction-fit pedicle screw assembly in accordance with an embodiment of the present disclosure.

FIG. 6B is a side view of a retainer ring for a friction-fit pedicle screw assembly in accordance with an embodiment of the present disclosure.

FIG. 6C is a top view of a retainer ring for a friction-fit pedicle screw assembly in accordance with an embodiment of the present disclosure.

FIG. 6D is a cross-sectional view of a retainer ring for a friction-fit pedicle screw assembly in accordance with an embodiment of the present disclosure.

FIG. 7A is a perspective view of a set screw for a friction-fit pedicle screw assembly in accordance with an embodiment of the present disclosure.

FIG. 7B is a top view of a set screw for a friction-fit pedicle screw assembly in accordance with an embodiment of the present disclosure.

FIG. 7C is a cross-sectional view of a set screw for a friction-fit pedicle screw assembly in accordance with an embodiment of the present disclosure.

FIG. 8A is a perspective view of a bone screw for a friction-fit pedicle screw assembly in accordance with an embodiment of the present disclosure.,

FIG. 8B is a cross-sectional view of the bone screw of FIG. 8A.

FIG. 8C is a top view of the bone screw of FIG. 8A.

FIG. 9A is a perspective view of a bone screw for a friction-fit pedicle screw assembly in accordance with an embodiment of the present disclosure.

FIG. 9B is a cross-sectional view of the bone screw of FIG. 9A.

FIG. 9C is a top view of the bone screw of FIG. 9A.

FIG. 10A is a perspective view of a bone screw for a friction-fit pedicle screw assembly in accordance with an embodiment of the present disclosure.

FIG. 10B is a cross-sectional view of the bone screw of FIG. 10A.

FIG. 10C is a top view of the bone screw of FIG. 10A.

FIG. 10D is a plan view of a bone screw for a friction-fit pedicle screw assembly in accordance with an embodiment of the present disclosure.

FIG. 11A is a cross-sectional view of an unassembled receiver in accordance with an embodiment of the present disclosure.

FIG. 11B is a cross-sectional view of a partially assembled receiver in accordance with an embodiment of the present disclosure.

FIG. 11C is a cross-sectional view of an assembled receiver in accordance with an embodiment of the present disclosure.

FIG. 12A is a cross-sectional view of an unassembled pedicle screw assembly in accordance with an embodiment of the present disclosure.

FIG. 12B is a cross-sectional view of a partially assembled pedicle screw assembly in accordance with an embodiment of the present disclosure.

FIG. 12C is a cross-sectional view of a partially assembled pedicle screw assembly in accordance with an embodiment of the present disclosure.

FIG. 12D is a cross-sectional view of an assembled pedicle screw assembly in accordance with an embodiment of the present disclosure.

FIG. 13A is a method of treating a spinal condition using the pedicle screw assembly in accordance with an embodiment of the present disclosure.

FIG. 13B is another method of treating a spinal condition using the pedicle screw assembly in accordance with an embodiment of the present disclosure.

FIG. 13C is another method of treating a spinal condition using the pedicle screw assembly in accordance with an embodiment of the present disclosure.

FIG. 14 is a method of assembling a spinal screw assembly in accordance with an embodiment of the present disclosure.

FIG. 15 is a method of assembling a receiver in accordance with an embodiment of the present disclosure.

FIG. 16 is another method of assembling a receiver in accordance with another embodiment of the present disclosure.

FIG. 17A is a perspective view of a pedicle screw assembly in accordance with an embodiment of the present disclosure.

FIG. 17B is a plan view of a pedicle screw assembly in accordance with an embodiment of the present disclosure.

FIG. 18A is a perspective view of a pedicle screw assembly in accordance with an embodiment of the present disclosure.

FIG. 18B is a partial cross-sectional, plan view of a pedicle screw assembly in accordance with an embodiment of the present disclosure.

FIG. 19A is a plan view of a tulip inserter (also referred to as an inserter tool) in accordance with an embodiment of the present disclosure.

FIG. 19B is a perspective view of a distal end of a tulip inserter in accordance with an embodiment of the present disclosure.

FIG. 19C is a partial cross-sectional, plan view of a portion of a tulip inserter engaged on a pedicle screw assembly in accordance with an embodiment of the present disclosure.

FIG. 20 is a method of using a tulip inserter to pre-lock a pedicle screw assembly in accordance with another embodiment of the present disclosure.

FIG. 21A is a plan view of a portion of a tulip inserter engaged on a pedicle screw assembly in accordance with an embodiment of the present disclosure.

FIG. 21B is a partial cross-sectional, plan view of a tulip inserter engaged on a pedicle screw assembly in accordance with an embodiment of the present disclosure.

FIG. 22A is a plan view of a portion of a tulip inserter engaged on a pedicle screw assembly in accordance with an embodiment of the present disclosure.

FIG. 22B is a partial cross-sectional, plan view of a tulip inserter engaged on a pedicle screw assembly in accordance with an embodiment of the present disclosure.

FIG. 23A is a plan view of a portion of a tulip inserter and a pedicle screw assembly in accordance with an embodiment of the present disclosure.

FIG. 23B is a plan view of a tulip inserter engaged on a pedicle screw assembly in accordance with an embodiment of the present disclosure.

FIG. 23C is a plan view of a tulip inserter engaged on a pedicle screw assembly with a distal plunger engaged in accordance with an embodiment of the present disclosure.

FIG. 24A is a perspective view of a retainer ring in accordance with an embodiment of the present disclosure.

FIG. 24B is a top view of a retainer ring in accordance with an embodiment of the present disclosure.

### DETAILED DESCRIPTION

For the purpose of promoting an understanding of the principles of the present disclosure, reference will now be made to the implementations illustrated in the drawings and specific language will be used to describe them. It will nevertheless be understood that no limitation of the scope of the disclosure is intended. Any alterations and further modifications to the described devices, instruments, methods, and any further application of the principles of the present disclosure are fully contemplated as would normally occur to one skilled in the art to which the disclosure relates. In addition, this disclosure describes some elements or features in detail with respect to one or more implementations or figures, when those same elements or features appear in subsequent figures, without such a high level of detail. It is fully contemplated that the features, components, and/or steps described with respect to one or more implementations or figures can be combined with the features, components, and/or steps described with respect to other implementations or figures of the present disclosure. For simplicity, in some instances the same or similar reference numbers are used throughout the drawings to refer to the same or like parts.

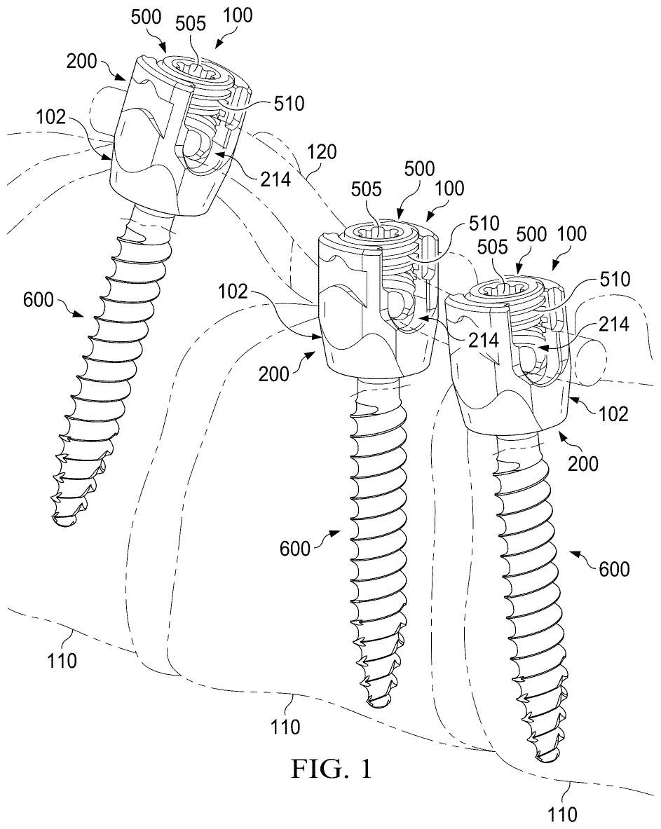

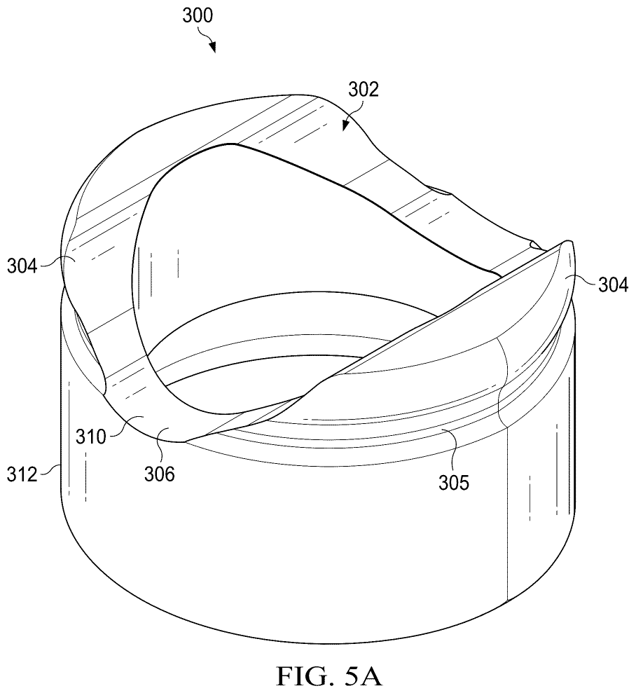

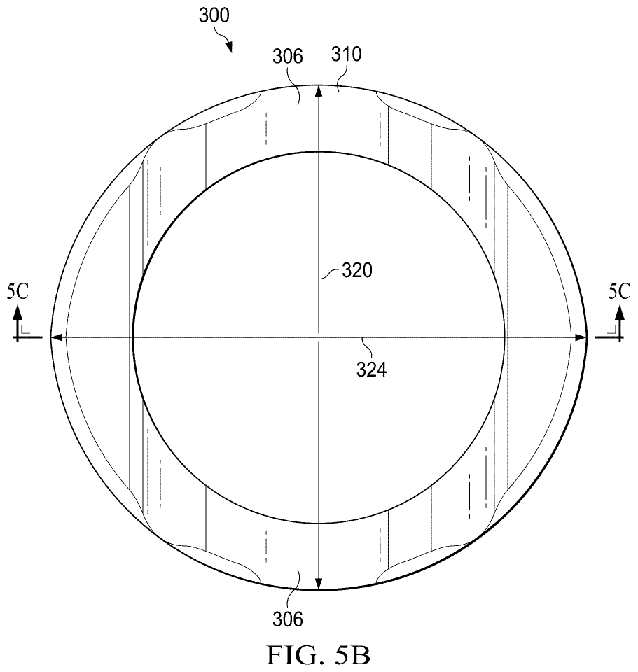

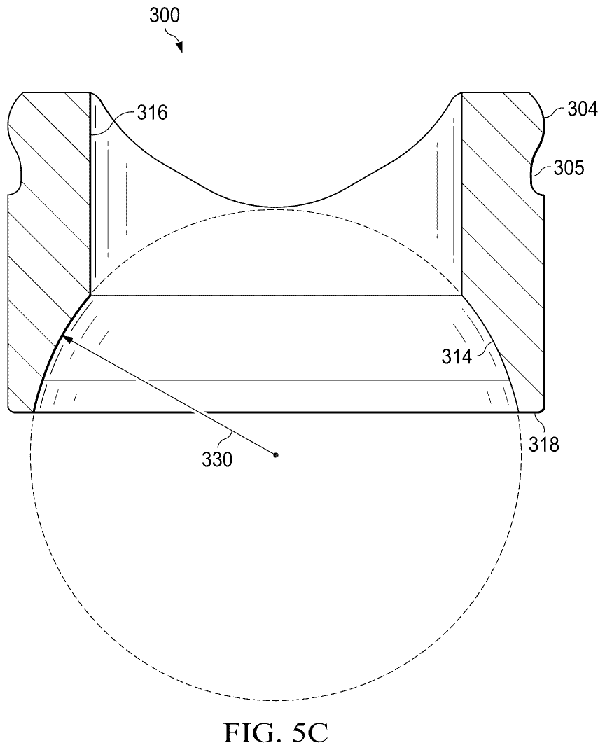

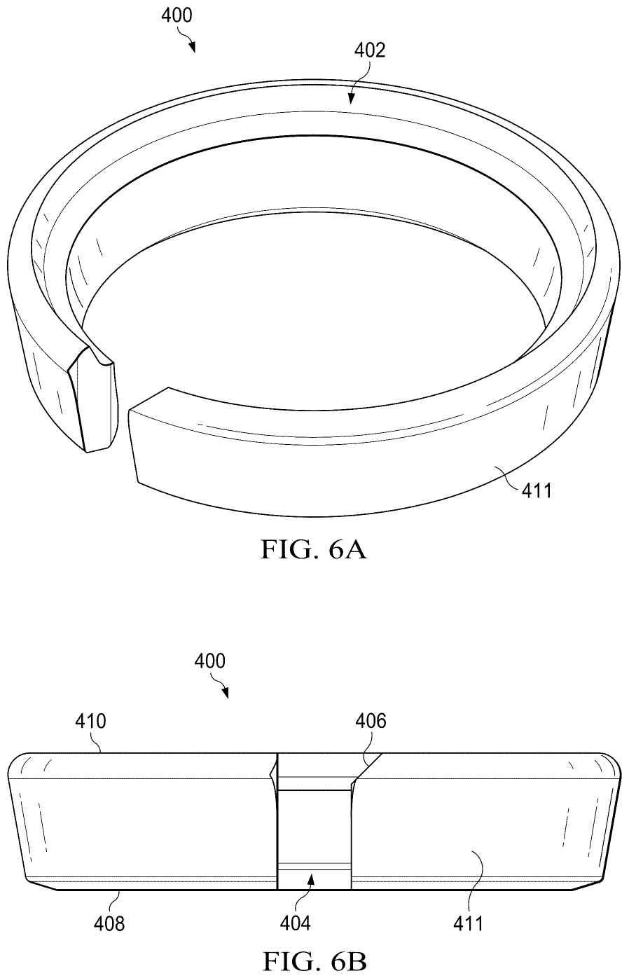

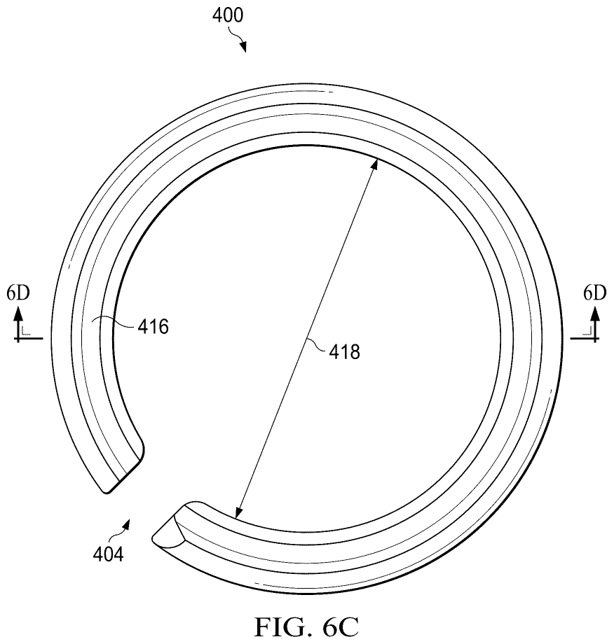

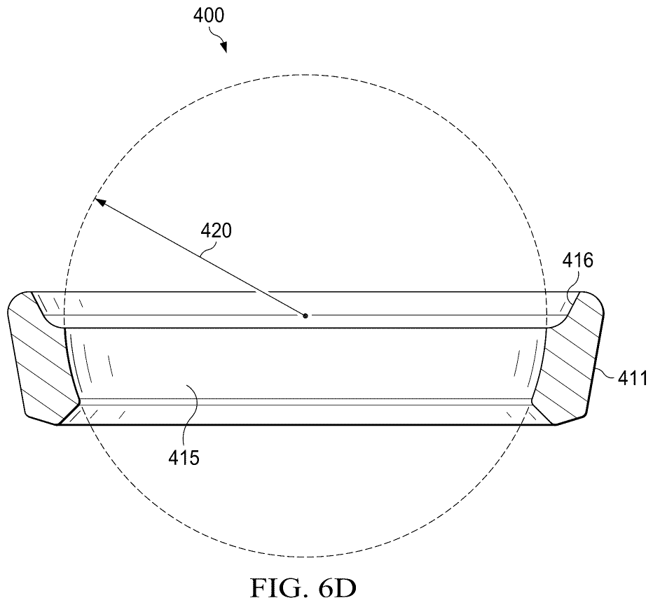

FIG. 1 is a perspective view of a plurality of pedicle screw assemblies 100 including a plurality of implantable receivers 102 coupled to respective vertebrae 110 of a patient's spine by a plurality of screws 600. Each implantable receiver 102 in FIG. 1 includes a body 200, and can further include a retainer ring (400, FIGS. 6A-6D or 440, FIGS. 24A and 24B) and a pressure cap (300, FIGS. 5A-5C) as described in more detail below. The receivers 102 are coupled to one another by a rod 120 positioned in U-shaped slots 214 or saddles of the receivers 102. The rod 120 can be sized, shaped (e.g., bent, curved), and otherwise structurally configured to correct a spinal deformity, and/or to retain the vertebrae 110 in a fixed position. The positions and orientations of the receivers 102 relative to the rod 120 and the bone screws 600 can be fixed or otherwise retained by the set screws 500. For example, the bone screws 600 can be coupled to the receivers 102 in a multi-axial relationship such that the bone screws 600 can be rotated about at least one axis relative to the respective receiver 102. For example, in some aspects, one or more of the bone screws 600 can include spherical, semi-spherical, or otherwise round screw heads (not shown) seated within the receiver 102. The receivers 102 can be configured to rotate, tilt, swivel, twist, and/or otherwise move relative to the screw heads of the bone screws 600. With the bone screws 600 fixed to the vertebrae 110, a physician can move the receivers 102 into the orientation shown in FIG. 1 to receive the rod 120. The orientations of the receivers 102 relative to one another can be maintained by the friction-fit coupling to the bone screws 600 as the physician guides the rod 120 through the slots 214. With the rod 120 in the slots 214, and with the desired overhang of the rod 120 from the outermost receivers 102, the set screws 500 can be tightened down to compress rod 120 and the screw heads of the screws 600 against the base of the receivers 102 to fix the position and orientation of the receivers 102 relative to the rod 120 and bone screws 600. The set screws 500 can be any appropriate shape. For example, the set screws 500 can include a drive feature 505 that receives an instrument for tightening the set screw 500 to compress the rod 120 and the screw heads of the screws 600. The drive feature 505 can be hexalobe shaped as shown in the illustrated embodiment, or can be any other appropriate shape including hexagonal, square, or triangular. Additionally, the set screws 500 can have threading 510 along part or all of the length. The threading 510 of the set screw 500 can threadably engage the receivers 102.

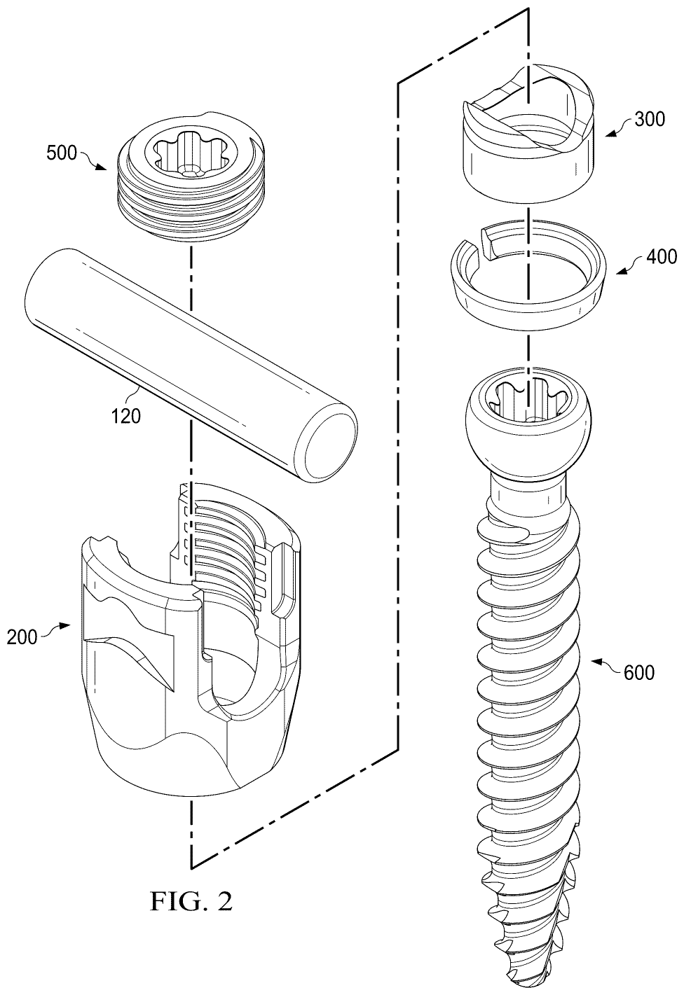

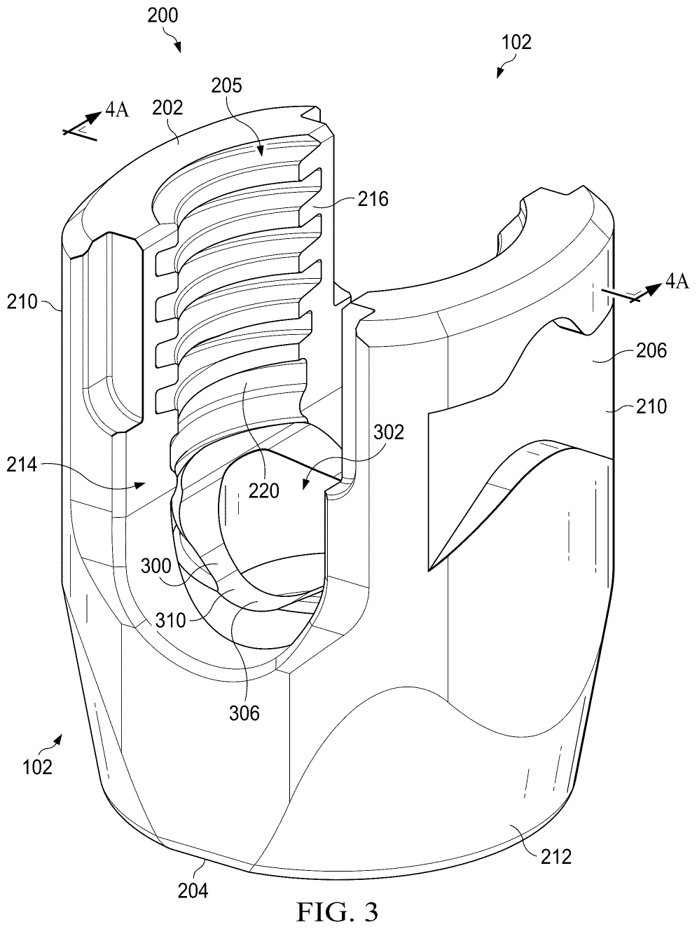

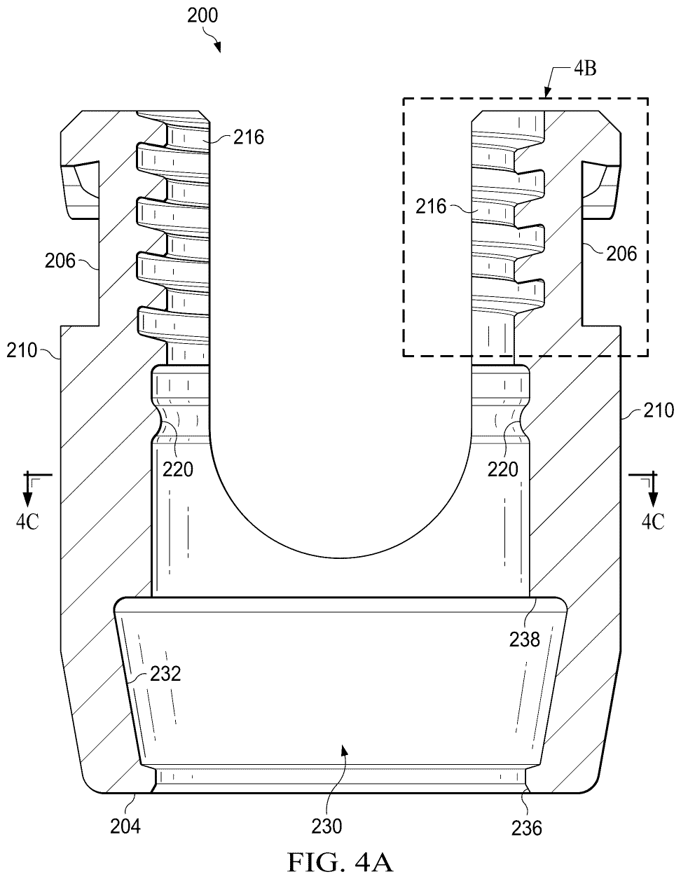

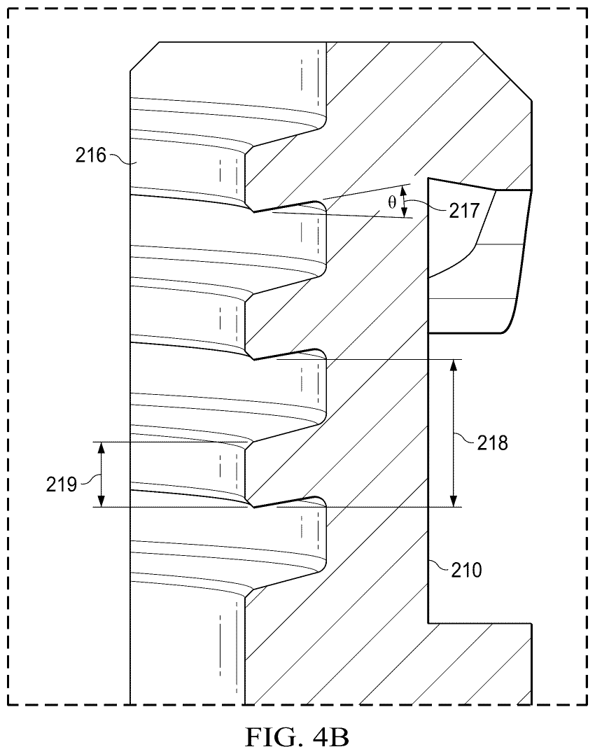

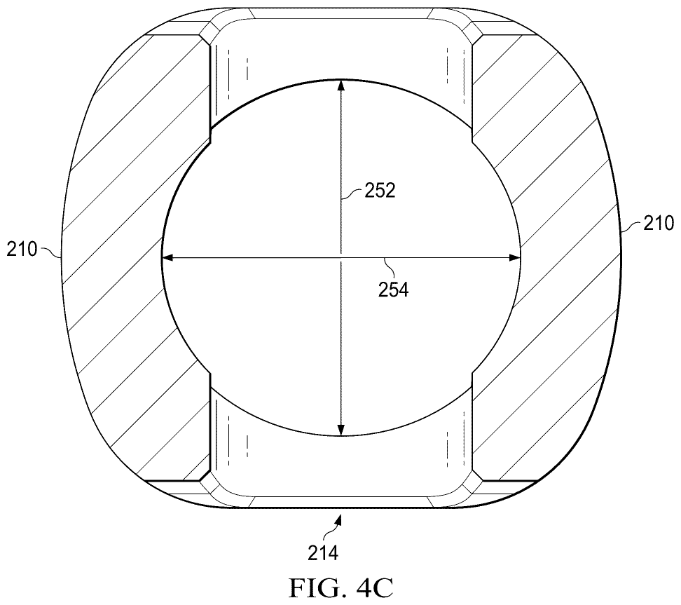

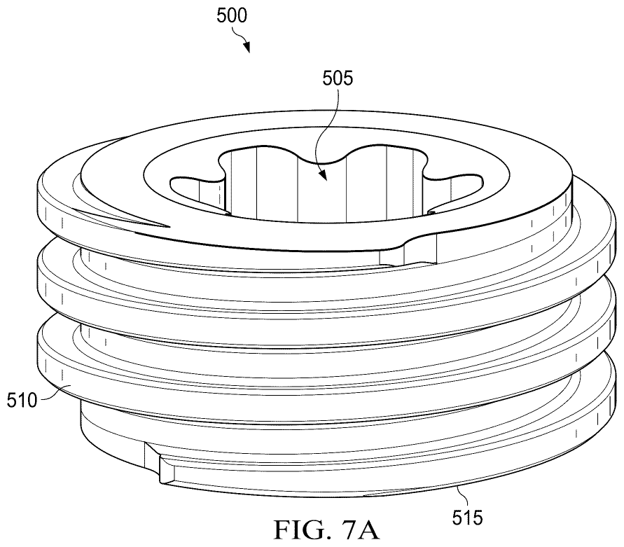

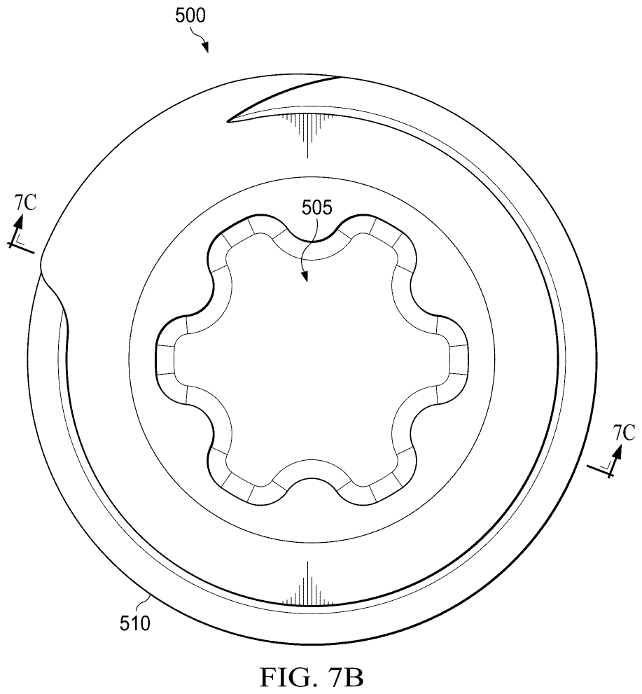

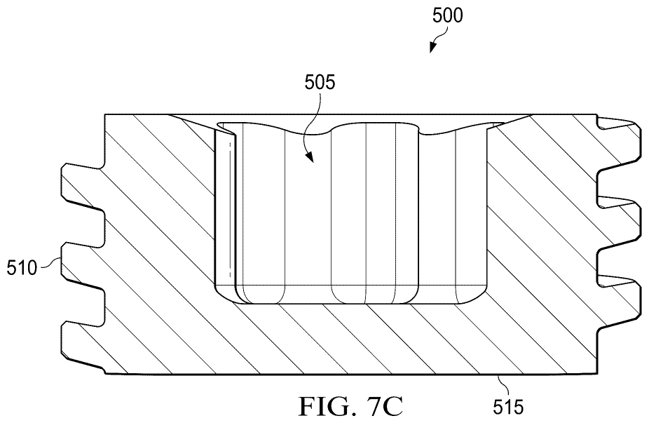

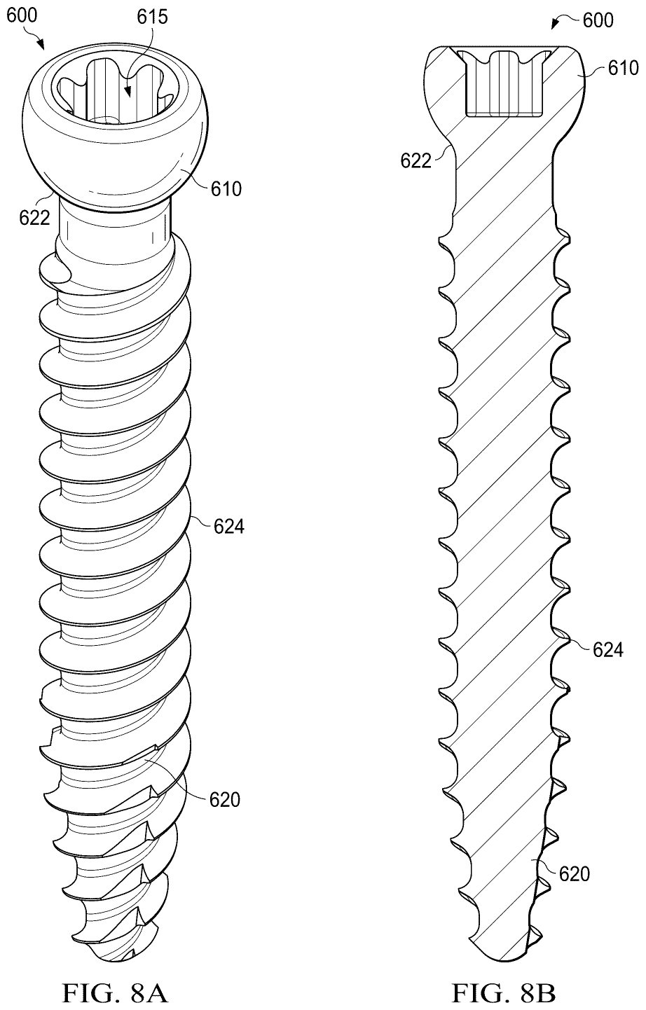

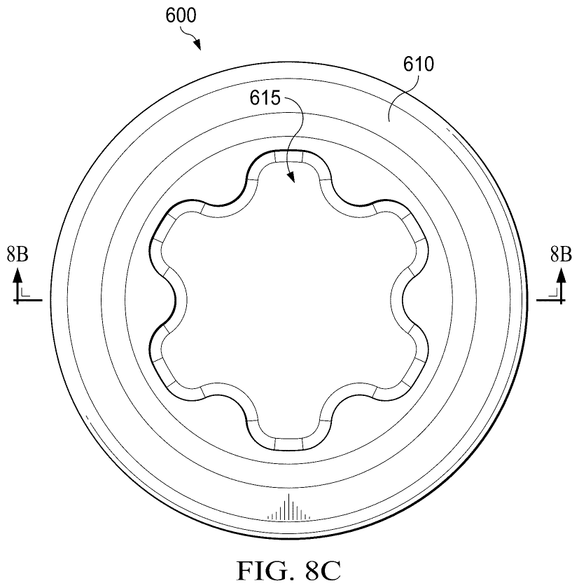

FIG. 2 is an exploded view of a friction-fit pedicle screw assembly 100 in FIG. 1. The screw assembly 100 includes a body 200, a pressure cap 300, a retainer ring 400, a screw 600, a rod 120, and a set screw 500. Body 200 is further described in FIGS. 3, 4A-4C and others below. Pressure cap 300 is further described in FIGS. 5A-5C and others below. Retainer ring 400 is further described in FIGS. 6A-6D and others below. Set screw 500 is further described in FIGS. 7A-7C. Various screws 600 are further described in FIGS. 8A-8C, 9A-9C, 10A-10D, and others below. It will be understood that the ordering of the components of the assembly 100 along the dashed lines may not necessarily dictate the order of assembly. In some aspects, however, the illustrated ordering in FIG. 2 may show the relative longitudinal positions of each component within the assembly. For instance, the threaded portion of the screw 600 can be the most distal component of the assembly 100, once assembled. The head of the screw 600 can partially overlap in the longitudinal direction with the retainer ring 400, which is positionable within the body 200 as shown below. The pressure cap 300 also is coupled to, and positionable within, the body 200 proximally of the retainer ring 400. The rod 120 can be seated on top of, or above (proximal to) the pressure cap 300, with the set screw 500 disposed above (proximal to) the rod 120.

In an exemplary embodiment, assembly of the pedicle screw assembly 100 can be as follows. The pressure cap 300 is positioned within the axial bore of the body 200. The pressure cap 300 can be inserted through the top opening or the bottom opening of the body 200. The retainer ring 400 is inserted through the bottom opening of the body 200 and positioned within a tapered chamber, cavity, or bore of the body 200 as explained further below.

In some aspects, receiver 102, depicted in FIG. 1, comprises some of the components shown in the exploded view in FIG. 2. The receiver 102 the body 200, pressure cap 300, and retainer ring 400, assembled as explained above. In some aspects, the receiver 102 can be assembled in a factory or manufacturing facility and packaged in a sterile package for delivery to a physician, hospital, or medical clinic. Receiver 102 can be selected from several similar receiver assemblies, for instance based on their type, size, geometry, and indication. Prior to or during surgery, the physician can select a screw 600 having the desired size (e.g., length) and characteristics (e.g., thread pitch, thread type, flutes, etc.) for the patient. The physician, or another technician present during the surgery, inserts the bulbous head of the screw 600 into the receiver 102 assembly. However, in other examples, the physician first inserts the screw 600 into the bone, and puts the receiver 102 onto the bulbous head of the screw 600 after the screw 600 is inserted. This configuration, in which a receiver 102 and a screw 600 can be selected and assembled by the physician prior to or during surgery, can be referred to as a “modular” system. The modularity of the assembly 100 advantageously allows for greater variety of components to fit the specific indications and anatomy of the patient. Modular systems can further reduce the amount of inventory the clinic, hospital, or physician maintains in order to have a suitable variety of assembly 100 sizes and types.

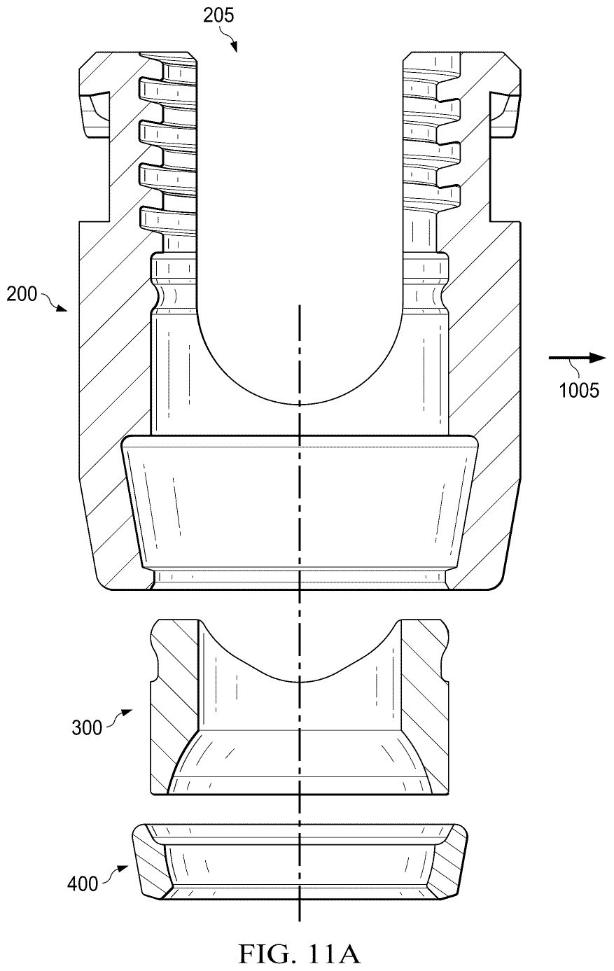

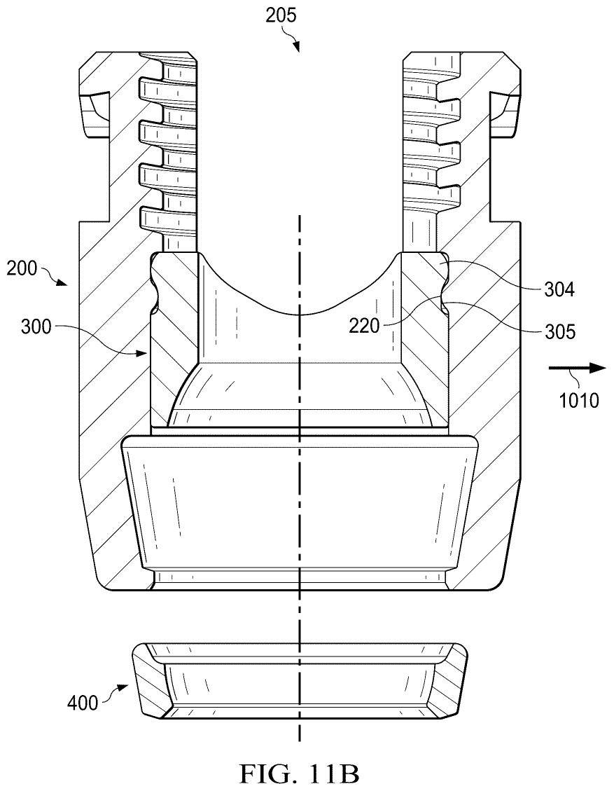

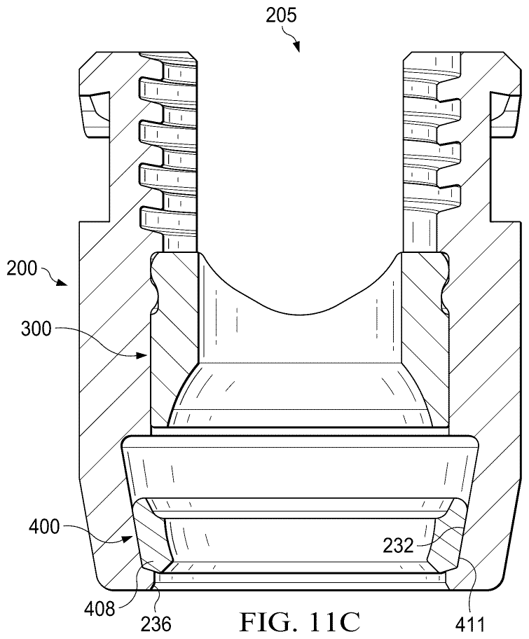

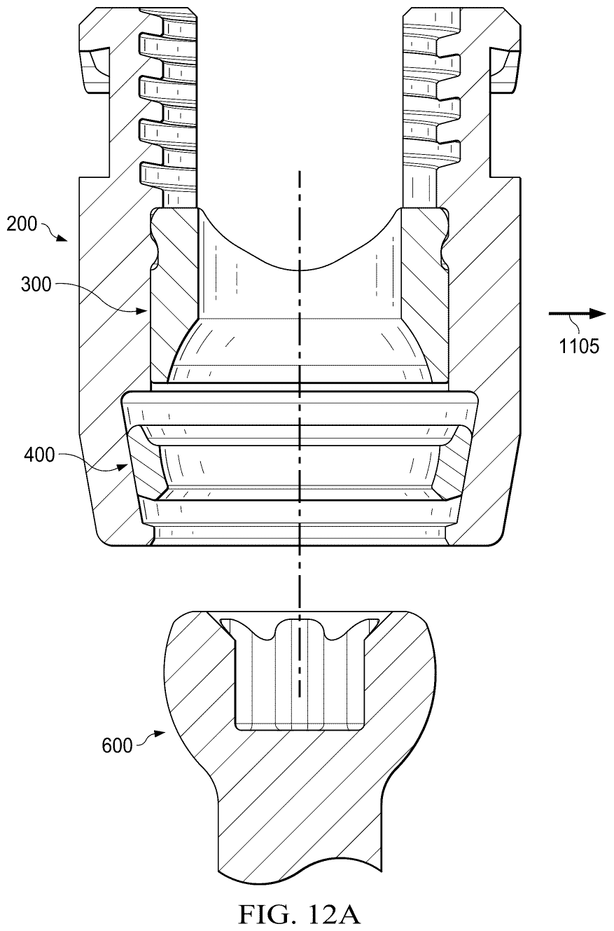

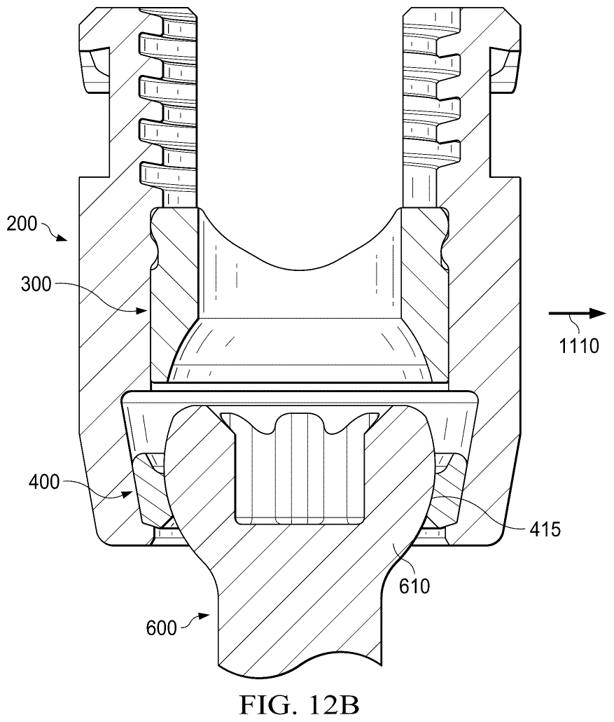

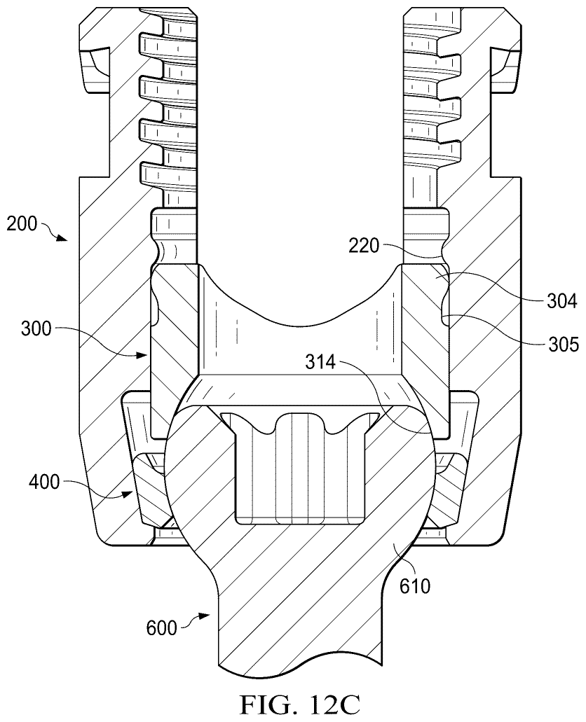

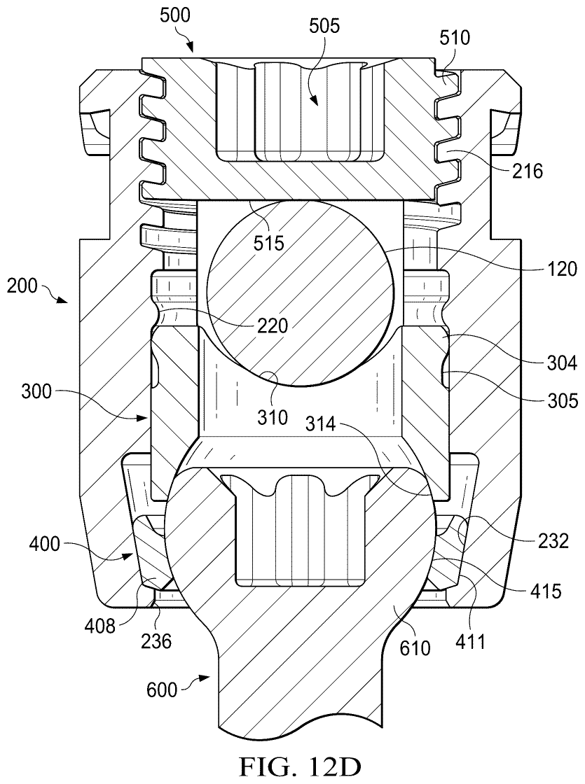

A method for assembling receiver 102 is further described in FIGS. 11A-11C. A method for assembling a portion of the screw assembly 100, e.g., screw 600 and receiver 102, is further described in FIGS. 12A-12D.

FIG. 3 is a perspective view of receiver 102 according to an embodiment of the present disclosure. In some aspects, the embodiment of the receiver 102 shown in FIG. 3 can be similar or identical to the receivers 102 shown in FIG. 1. The receiver 102 includes a body 200, a retainer ring 400 or lock ring, and a pressure cap 300. The body 200 has a top end 202 for receiving a set screw (e.g., 500, FIG. 1) and a rod (e.g., 120, FIG. 1) and a bottom end 204 for receiving a screw head. The body 200 can further comprise an opening 205. The opening 205 can pass from the top end 202 to the bottom end 204. In the illustrated embodiment, body 200 is tulip-shaped, meaning the body 200 has two arms 210 on either side of the body 200 that extend from a base 212 of the body 200 to the top 202. Arms 210 define a channel or U-shaped slot 214 for seating a rod. Depending on the implementation, some examples of the U-shaped slot 214 have a width sized to receive a spinal rod. In some example implementations the U-shaped slot 214 has a width within a range of about 3 mm to 9 mm, although other widths are contemplated. In some examples, the width is in a range of about 5 mm to 7 mm. Arms 210 can be referred to as sidewalls, wings, or any other suitable term. Body 200 is configured to receive a connecting rod via the U-shaped slot 214. Moreover, the body 200 further includes internal threads 216 on the interior surfaces of the arms 210. The threads 216 can be configured to engage corresponding threads on a set screw (e.g., 500, FIG. 1). The set screw can be tightened down into the body 200 to compress the connecting rod onto the pressure cap 300. Compressing the pressure cap 300 can also cause the pressure cap 300 to put additional pressure onto the screw head 610 of the screw 600 to fix the receiver 102 in a desired position and orientation.

The body 200 also has two engagement features 206 that can provide for releasable engagement with a tool for inserting, positioning, and/or removing the receiver 102. For example, the engagement features 206 can provide for releasable engagement with a tool for inserting the subassembly including the receiver 102 and the connected screw 600, and driving the screw 600 into the patient's bone (e.g., vertebra). In the illustrated embodiment, the engagement feature 206 is centered with the arm 210. It will be understood that the other arm 210 can also include an engagement feature similar or identical to the engagement feature 206. The engagement feature 206 on the other arm 210 can also be centered on the arm 210. The centering of the engagement feature 206 can be beneficial for robust engagement with the insertion tool. For example, the centered placement of the engagement feature 206 can allow for a deeper groove or impression of the engagement feature 206 into arm 210. In another aspect, the top end 202 of the body 200 can be associated with a frangible portion or breaking line of the body 200. For example, in some embodiments, the body 200 can be integrally formed with extension portions or tower portions extending proximally from the top end 202. The area of the body 200 comprising the top end 202 can comprise a weakened portion.

The receiver 102 also includes a pressure cap 300, which can also be referred to as a pressure member or saddle. The pressure cap 300 includes a concave upper surface or top surface for receiving the connecting rod, as described above. The pressure cap 300 can be saddle-shaped, meaning the pressure cap 300 has two ends 304 with an arched surface forming a depression 306 between the two ends 304. This saddle-shape can generally match and/or align with the shape of the U-shaped slot 214 formed between the arms 210 of the body 200. Thus, the pressure cap 300 can be shaped to accept a rod that is placed within the U-shaped slot 214 of the body 200. Further, the depression can provide additional relief for the pressure cap 300 to deform or flex upon locking when the set screw 500 is urged downward against the rod. The pressure cap 300 can also include a concave surface on the bottom side of the pressure cap 300 to contact and engage a top surface of a screw head. However, in other embodiments, the pressure cap 300 can have any appropriate shape having a top for seating a rod and a bottom for contacting a screw head. For example, the pressure cap 300 can include a v-shaped depression, a rectangular depression, an elliptical depression, a hexagonal depression, and/or any other suitable shape for receiving the connecting rod. Similarly, the bottom surface of the pressure cap 300 can be flat, inclined, saddle-shaped and can be shaped elliptically, rectangularly, hexagonally or any other suitable shape for contacting and engaging a top surface of a screw head. In some instances, the pressure cap can be undersized such that contact between the pressure cap and the screw head is a line (e.g., a circle) instead of a surface (e.g., an annular region of a spherical surface). For example, the radius of curvature 330, as depicted in FIG. 5C, can be smaller than the radius of the screw head.

The pressure cap 300 can include protrusions 304 near opposite sides of the top surface 310 (only one protrusion is visible in FIG. 3). Protrusions 304 interact with bump or detent 220 disposed circumferentially on the arms of the body 200, surrounding the opening 205. The detent can have a semicircular cross-section, where the cross-section is taken through the arms 210 of the body 200 in a plane parallel to the longitudinal axis of the body 200. During assembly of the receiver 102, the protrusions 304 and the detent can have one or more contact surfaces. When the pressure cap 300 is in a disengaged configuration, protrusions 304 can contact upper side of the detent 220, whereas in an engaged configuration with a screw head, protrusions can contact the bottom side of the detent 220. In the disengaged configuration, detent 220 can conform to and/or be in contact with a circumferential depression 305 on the outer surface of the pressure cap 300. The engagement or contact between the pressure cap 300 and the detent 200 can further involve a groove formed or defined in the outer surface of the protrusions 304, as explained below. The steps of assembling the receiver and inserting a bone screw 600 are further described below in FIGS. 11A-11C and 12A-12D.

Moreover, the pressure cap 300 has an opening 302 extending through the center and aligning with the opening 205 of the body 200. The opening 302 allows an instrument to access a head of a screw when it is inserted into the receiver 102. For example, an interfacing portion or bit of a screw driver can be able to pass through the opening 302 of the pressure cap 300 so that the bone screw can be screwed into bone.

The retainer ring 400 is located within a base 212 of the body 200 and will be described in more detail below.

In some embodiments, the receiver 102 can comprise a pin that is received in a pin hole in the side of the body 200. The pin projects into the opening 205 of the body 200. The pin can be welded, adhered, soldered, threadably attached, and/or otherwise affixed, attached, or coupled to the body 200. In other embodiments, the pin can be formed in the body 200. The pressure cap 300 can have a slot that is shaped to receive the pin. This allows the pressure cap 300 to move up and down along the opening 205 of the body 200, but minimizes the rotation of the pressure cap 300 so that it remains in a relatively constant orientation.

In some embodiments, the depression and protrusions can be switched. For example, the body can include a circumferential depression which conforms and/or contacts a detent located on the two sides of the pressure cap or circumferentially around the outer surface of the pressure cap.

The materials of the receiver 102 can be biocompatible, and can have other structural characteristics appropriate for use in spinal fixation. For example, the body 200, pressure cap 300, retainer ring 400, and/or the screw 600 can include a biocompatible metal, such as stainless steel, titanium, and/or alloys thereof. In other embodiments, one or more components of the receiver 102 can include a polymer material, such as DELRIN, polyether ether ketone (PEEK), polytetrafluoroethylene (PTFE), polysulfone (PS), polycarbonate, and/or any other suitable polymeric material. One or more components of the receiver 102 can be manufactured by milling, machining, casting, molding, laser sintering, 3D printing, and/or any other suitable process. The components of the receiver 102 can be formed of the same materials or of different materials.

FIGS. 4A-4C depict cross-sectional views of a body 200. FIG. 4A depicts a cross-section of a body 200. FIG. 4B depicts a magnified cross-sectional view of threading 216 of the body 200 as indicated in FIG. 4A. FIG. 4C is a cross-sectional view of body 200 as indicated in FIG. 4A. Body 200 is depicted without the pressure cap 300 or retainer ring 400.

Receiver body 200 has a top end 202 for receiving a set screw (e.g., 500, FIG. 1) and a rod (e.g., 120, FIG. 1) and a bottom end 204 for receiving a screw head. The receiver body 200 can further comprise an opening or axial bore or opening 205. The opening 205 can pass from the top end 202 to the bottom end 204. In the illustrated embodiment, the receiver body 300 is tulip-shaped, meaning the receiver body 200 has two arms 210 on either side of the receiver body 200 that extend from a base 212 of the receiver body 200 to the top 202. Arms 210 define a channel or U-shaped slot 214 for seating a rod. Arms 210 can be referred to as sidewalls, wings, or any other suitable term. Receiver body 200 is configured to receive a connecting rod via the U-shaped slot 214. Moreover, receiver body 200 further includes internal threads 216 on the interior surfaces of arms 210. The threads 216 can be configured to engage corresponding threads on a set screw (e.g., 500, FIG. 1). In some instances, the arms 210 of the receiver body 200 include inverted buttress threading as characterized by an angle θ 217, where θ=0° indicated no inverted buttress. The angle θ 217 can be any number different values from 0° to 60°. In some instances, an angle of the lower surface of a thread and an upper surface of a thread can differ. For example, the angle of an upper surface of a thread can be 10°, while the angle of a lower surface of a thread can be 15°. Threading can have a pitch 218 and a separation distance 219 between threads determined by the application/intervention.

The receiver body 200 also has two engagement features 206 that can provide for releasable engagement with a tool for inserting, positioning, and/or removing the receiver 102. For example, the engagement features 206 can provide for releasable engagement with a tool for inserting the subassembly including the receiver 102 and the connected screw 600, and driving the screw 600 into the patient's bone (e.g., vertebra). In the illustrated embodiment, engagement feature 206 is centered with the arm 210. It will be understood that the other arm 210 can also include an engagement feature similar or identical to the engagement feature 206. The engagement feature 206 on the other arm 210 can also be centered on the arm 210. The centering of the engagement feature 206 can be beneficial for robust engagement with the insertion tool. For example, the centered placement of the engagement feature 206 can allow for a deeper groove or impression of the engagement feature 206 into the arm 210. In another aspect, the top end 202 of the receiver body 200 can be associated with a frangible portion or breaking line of the receiver body 200. For example, in some embodiments, the receiver body 200 can be integrally formed with extension portions or tower portions extending proximally from the top end 202. The area of the receiver body 200 comprising the top end 202 can comprise a weakened portion.

The receiver body 200 further comprises a ridge or detent 220 formed in an interior surface of the receiver body 200. In the illustrated embodiment, the detent includes two portions on either side of a U-shaped slot 214 of the receiver body 320, in which the connecting rod is received. Detent 220 can conformably fit to the depression 305 on the outer surface of pressure cap 300. Detent 220 can have a semi-circular cross-section. In some embodiments, detent 220 can have other cross-sections including all or portions of a square, wedge, triangle, elliptical, etc. Detent 220 can extend about an entirety of the interior surface surrounding the axial bore of the receiver 102, but separated or interrupted by the rod channel 214. In other examples, the detent 220 can comprise multiple separate ridges, bumps, or other protrusions. The ridge or detent 220 is shown as having a symmetrical cross section which is rounded both on its top side and bottom side. This rounding on both sides can facilitate selective engagement, disengagement, and reengagement of the pressure cap's projections 304.

As shown in FIG. 4C, the axial bore of the receiver body can have an elliptical cross-section. The elliptical cross section includes a first axis 252 and second axis 254. The orientation of the elliptical cross-section is configured such that a pressure cap 300 is in fixed orientation about the axis defined by the opening 205. In some aspects, the relative size of the first axis and second axis can be interchanged, i.e., the orientation of the ellipse, defined by either of the axes, relative to the rod channel can differ. Said another way, in some instances the first axis will be shorter than the second axis, whereas in other instances the first axis will be longer than the second axis. In some examples, the first axis can have a length in a range of about 5 mm to 12 mm, although other lengths are contemplated. In some examples, the length is in a range of about 7 mm to 8 mm. In some examples, the second axis can have a length in a range of about 5 mm to 12 mm, although other lengths are contemplated. In some examples, the length is in a range of about 7 mm to 8 mm. In some examples, the length of the first axis is about 8.29±0.3 millimeters and the second axis can be 8.79±0.3 millimeters. However, other lengths can be used for the first and second axes, e.g., the first and second axes can be between 1 millimeter and 15 millimeters.

FIGS. 5A-5C depict views of a pressure cap 300. FIG. 5A is a perspective view of pressure cap 300. FIG. 5B depicts a top view of pressure cap 300 as depicted in the perspective view of FIG. 5A. FIG. 5C is a cross-sectional view of pressure cap 300.

The receiver 102 includes a pressure cap 300, which can also be referred to as a pressure member, a pressure insert, or saddle. The pressure cap 300 includes a concave upper surface or top surface 310 for receiving the connecting rod, as described above. The pressure cap 300 can be saddle-shaped, meaning the pressure cap 300 has two ends 304 with an arched surface forming a depression 306 between the two ends 304. This saddle-shape can generally match the shape of the U-shaped slot 214 formed between the arms 210 of the receiver body 200. Thus, the pressure cap 300 can be shaped to accept a rod that is placed within the U-shaped slot 214 of the receiver body 200. The pressure cap 300 can also include a bottom inner wall 314 having a concave surface on the bottom side of the pressure cap 300 to contact and engage a top surface of a screw head. The concave inner surface can have a radius of curvature 330. In some embodiments, the radius of curvature can be between 1 and 10 millimeters. For example, the radius of curvature can be 4 millimeters in the depicted embodiment. However, in other embodiments, the pressure cap 300 can have any appropriate shape having a top for seating a rod and a bottom for contacting a screw head. For instance, the radius of curvature 330 can be smaller than the radius of the spherical part of the screw head, or can be the same radius. The inner wall 314 can be configured to flex outward. There can be vertical cuts or slots in the outer wall of the insert through the inner wall 314 to allow the bottom inner wall 314 to flex, creating a spring force that is applied to the screw head when assembled. For example, the pressure cap 300 can include a v-shaped depression, a rectangular depression, an elliptical depression, a hexagonal depression, and/or any other suitable shape for receiving the connecting rod. Similarly, the bottom surface of the pressure cap 300 can be flat, inclined, saddle-shaped and can be shaped elliptically, rectangularly, hexagonally or any other suitable shape for contacting and engaging a top surface of a screw head. The top inner wall 316 of the pressure cap 300 can be cylindrical.

Moreover, the pressure cap 300 has an opening 302 extending through the center and aligning with the axial bore 205 of the receiver body 200. The opening 302 allows an instrument to access a head of a screw when it is inserted into the receiver 102. For example, an interfacing portion or bit of a screw driver can be able to pass through the opening 302 of the pressure cap 300 so that the bone screw can be screwed into bone.

The two ends 304 of the pressure cap 300 can be a pair of protrusions extending outward from an outer surface of the pressure cap 300. The protrusions 304 can alternatively be referred to as wings, projections, lips, or any other suitable term. The protrusions 304 can be integrally formed with the pressure cap 300, or can comprise separate components or elements that are attached, fixed, or otherwise connected to the body of the pressure cap 300. For instance, the protrusions 304 can be adhered, welded, or press fit into a corresponding recess or surface in the body of the pressure cap 300. In another example, the protrusions 304 can be machined from a monolithic or integral structure that forms the body of the pressure cap 300. The protrusions 304 can be formed by a combination of machining and permanent plastic deformation. Protrusions 304 can be adjacent to a circumferential depression 305 on the outer surface of the pressure cap 300. Depression can be semi-circular and configured to interact with the detent 220 located on the inner surface of the body 200. In some embodiments, the protrusions may not be synonymous with the ends 304. For example, the protrusions can be located higher or lower on the outer surface of the pressure cap 300 (i.e., more proximal or distal, respectively) or at different circumferential positions (including different numbers of positions, e.g., 1, 2, 3, 4, and up to 20) around the outer surface of the pressure cap 300.

The pressure cap 300 can have protrusions, also called wings or arms, that extend upward and a groove/depression 305. In other words, the arms/wings may not extend radially outward from the pressure cap 300.

As shown in FIG. 5B, the pressure cap 300 can have an elliptical shape defined by a third axis 320 and fourth axis 324. The third axis 320 and fourth axis 324 are configured to be smaller than the first axis 252 and second axis 254 of the body 200, but close enough in size to restrict the rotation of the pressure cap 300 within the body 200 about the axis defined by the axial bore. In some aspects, the relative size of the third axis 320 and fourth axis 324 can be interchanged, i.e., the orientation of the ellipse, defined by either of the axes, relative to the rod channel can differ. Said another way, in some instances the third axis 320 will be shorter than the fourth axis 324, whereas in other instances the third axis 320 will be longer than the fourth axis 324. In one example, the third axis 320 can be 8.14±0.3 millimeters and the fourth axis 324 can be 8.64±0.3 millimeters. In this example and comparing with the example given above for the first and second axis, the pressure cap 300 will have its fourth axis 324 approximately aligned with the second axis of the body 200 and the third axis 320 with the first axis of the body. However, other lengths can be used for the third and fourth axes (320 and 324, respectively), e.g., the third and fourth axes (320 and 324, respectively) can be between 1 millimeter and 15 millimeters.

The protrusions 304 can be configured to flex inward by application of a force such that the protrusions 304 elastically deform in a spring-like fashion. As will be explained in more detail below, the protrusions 304 are sized, shaped, and otherwise structurally configured to be positioned above or below the detent 220 of the body 200. The size and geometry of the protrusions 304 is such that the protrusions 304 contact and interfere with the detent 220 longitudinal moving the pressure cap 300 relative to the body 200. As the pressure cap 300 moves longitudinally upward from below the detent 220, or approximately, relative to the receiver body 200, the surfaces of the detent 220 cause the protrusions 304 to flex inward. In other words, detent 220 applies a force against the motion of the pressure cap 300, such that a sufficient force causes a “click” between two different configurations of the pressure cap 300 within the body 200. Similarly, sufficient downward force can be applied to the pressure cap 300 to move it past the detent 220 and engage the head of a bone screw which has been inserted into the receiver 102.

FIGS. 6A-6D depict views of a retainer ring 400. FIG. 6A is a perspective view of retainer ring 400. FIG. 6B is a side view of retainer ring 400. FIG. 6C is a top view of retainer ring 400. FIG. 6D is a cross-sectional view of retainer ring 400.

When the receiver 102 is assembled, the retainer ring 400 is located around the axial bore 205 proximate the bottom 204 of the body 200. In this embodiment, the retainer ring 400 is a split ring having a gap 404 that has a discontinuous annular shape configured to expand and/or retract to enlarge and/or reduce an inner diameter of the retainer ring 400. In other embodiments, the retainer ring 400 can be a continuous ring capable of expanding over a screw head when it is inserted from the bottom 204 of the body 200. The retainer ring 400 can be configured to lock the screw 600 into the receiver 102 once the screw head 210 has been inserted through a bottom opening of the retainer ring 400, as shown in FIGS. 4B-4D, for example. In some embodiments, the upper surface of the screw head 210 can be spherical, rounded, tapered, or otherwise configured to cause the retainer ring 400 to expand as the screw head 610 is pressed against the retainer ring 400, e.g., at or near the ledge 416, to allow the screw head 610 to pass through the retainer ring 400. Once the screw head 210 has passed through the retainer ring 400, the retainer ring 400 can relax and contract to lock against a bottom curved surface of the screw head 610, e.g., screw head 610 can rest against inner surface 415 of the retainer ring 400. In some embodiments, an inner surface 415 of the retainer ring 400 includes a concave ridge or seating feature configured to engage the bottom surface of the screw head 210. The inner surface 415 can have a spherical annular surface with a radius of curvature 420. In some embodiments, the radius of curvature 420 can be between 1 and 10 millimeters. For example, the radius of curvature 420 can be 4 millimeters in the depicted embodiment. However, in other embodiments, the receiver 102 can have any appropriate component that locks the screw head 610 into the receiver 102, such as spring-loaded ball bearings, yielding locking ridge, and/or any other suitable feature.

In some embodiments, ledge 416 allows the pressure cap 300 to be positioned or rest more distally within the body. In some instances, ledge 416 allows improved contact of the pressure cap 300 with screw 600. The shape of the retainer ring 400 including ledge 416 can facilitate a more rigid structure.

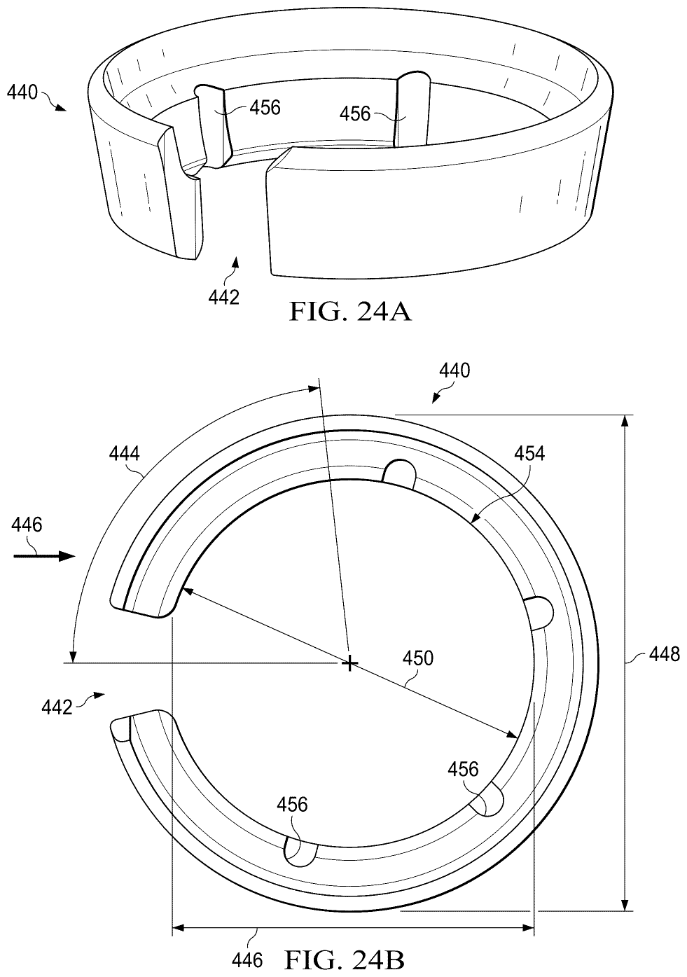

FIGS. 24A and 24B depict views of an alternative retainer ring 440. FIG. 24A is a perspective view of retainer ring 440. FIG. 24B is a top view of retainer ring 440. Although this specification describes the implantable receiver 102 as having the example retainer ring 400, the implantable receiver 102 can include the retainer ring 440 described with reference to FIGS. 24A and 24B in any of the embodiments herein. The retainer ring 440 includes many of the same shapes and features as the retainer ring 400 as would be apparent to one of ordinary skill in the art, and therefore not all those features are repeated. However, some of the differences are set out in the discussion below with reference to FIGS. 24A and 24B.

In one or more embodiments, the retainer ring 440 contains a split portion 442 (or gap) that interrupts the circumference of the retainer ring 440 on one side, as shown in FIG. 24B. In some examples, the split portion 442 or gap may align with an angle in a range of about 1 to 90 degrees of the circumference of the retainer ring, although other angles are contemplated. In some examples, the angle is in a range of about 15 to 45 degrees. In yet other examples, the angle is in a range of about 25-35 degrees. The retainer ring 440 has an inner diameter 450 and an outer diameter 448. The inner diameter 450 may be selected to sufficiently interact with the screw head, and the outer diameter may be selected to interact with the opening in the bottom of the receiver. Depending on the implementation, the inner diameter 450 may be in a range of about 3 mm to 15 mm, although other larger and smaller diameters are contemplated. In some implementations, the inner diameter 450 is in a range of about 7 mm to 8 mm. The outer diameter 448 can be in a range of about 5 mm to 35 mm, although other larger and smaller diameters are contemplated. In some implementations, the outer diameter 448 is in a range of about 10 mm to 11 mm.

A ring bend section 444 disposed at an end adjacent the split portion 442 can be pushed (i.e., bent) inward toward the center of the retainer ring, as indicated by the direction of arrow 446. The bend section radius 454 is correspondingly changed as ring bend section 444 is bent inward. The ring bend section 444 may be in a range of about 10 to 180 degrees of the circumference of the ring, although other angles are contemplated. In some examples, the angle is in a range of about 45 to 120 degrees. In yet other examples, the angle is in a range of 75 to 90 degrees.

A plurality of example relief cuts 456 are shown in FIGS. 24A and 24B. In the example shown, the relief cuts 456 are shown as arcs or arc-shaped. In other implementations, the relief cuts 456 may also or alternatively be shaped as notches, V-shaped cuts, and the like, or any combination thereof. Relief cuts 456 may also be increased or decreased in number and/or in depth of cut into the retainer ring 440, as appropriate. The example shown includes four relief cuts 456 spaced at 60 degree intervals. In one or more embodiments, the relief cuts 446 allow the retainer ring 440 to more easily elastically deform instead of plastically deform upon installation and/or assembly of the retainer ring to the receiver body 200, and upon installing the body 200 onto the screw 600 where the retainer ring 440 in the body 200 is expanding to accept the screw head 610 therethrough. In an embodiment, the relief cuts 456 are not evenly distributed so that the “bend zone” (e.g., the ring bend section 444) does not coincide with a relief cut 456. This can reduce the likelihood of potentially weakening the integrity of the retainer ring.

In one or more embodiments, the retainer ring 440 is bent on one side only, making the inner diameter 450 of the retainer ring 440 smaller, allowing the retainer ring 440 to push or maintain the screw head 610 in an upward state, making the space between the screw head 610 and the pressure cap 300 smaller, thereby creating some amount of friction. Although the ring bend section 440 can be deformed in any way, in some implementations, the retainer ring 440 is fixed in a fixture and a plunger is used to push the ring bend section 440 inward in the direction shown by the arrow 446. This results in one side of the retainer ring being bent inward. Since the one side is bent inward, the plastic deformation of the ring changes the diameter, when measured at the ring bend section, and therefore also changes the relaxed width 446 of the retainer ring 440.

In some implementations, the retainer ring 400 includes features described with reference to the retainer ring 440 (FIGS. 6A-6D). For example and without limitation, some implementations of the retainer ring 400 includes a ring bend section similar to the ring bend section 444 described herein and with reference to FIG. 24B. Other modifications would also be apparent to one of ordinary skill in the art.

FIGS. 7A-7C depict views of a set screw 500. FIG. 7A is a perspective view of set screw 500. FIG. 7B is a top view of set screw 500. FIG. 7C is a cross-sectional side view of set screw 500. The set screws 500 can be any appropriate shape. For example, the set screws 500 can include a drive feature 505 that receives an instrument for tightening the set screw 500 such that the bottom surface 515 contacts the rod 120 to compress the rod 120 and the screw heads of the screws 600. The drive feature 505 can be hexalobe shaped as shown in the illustrated embodiment, or can be any other appropriate shape including hexagonal, square, or triangular. Additionally, the set screws 500 can have threading 510 along part or all of the length. The threading 510 of the set screw 130 can threadably engage the receiver 102.

In some instances, the threading as characterized by an angle θ similar to the angle depicted in FIG. 4B, where θ=0° indicated no inverted buttress. The angle θ can be any number of different values from 0° to 60°. In some instances, an angle of the lower surface of a thread and an upper surface of a thread can differ. For example, the angle of an upper surface of a thread can be 10°, while the angle of a lower surface of a thread can be 15°. Threading can have a pitch and a separation distance between threads determined by the application/intervention.

FIGS. 8A-8C depict views of a first bone screw 600 for use in pedicle screw assembly 100. FIG. 8A is a perspective view of a first bone screw 600. FIG. 8B is a cross-sectional view of first bone screw 600. FIG. 8C is a top view of first bone screw 600. In the illustrated embodiment, the dual lead pitch screw 600 comprises a head 610 having a spherical-shaped bottom 622 and a shaft 620. Shaft 620 comprises threading 624 that extends completely or partially down the length of the shaft 620. The threading 624 of the dual lead pitch screw comprises two starts. The dual lead pitch screw 600 can be advantageous for use in cortical bone. However, in other embodiments, the screw 600 may not be dual lead and instead can have one start or more than two starts. Moreover, the screw 600 can have any appropriate pitch or lead. Screw 600 includes a drive feature 615 which can be hexalobe shaped as shown in the illustrated embodiment, or can be any other appropriate shape including, but not limited to, hexagonal, square, or triangular.

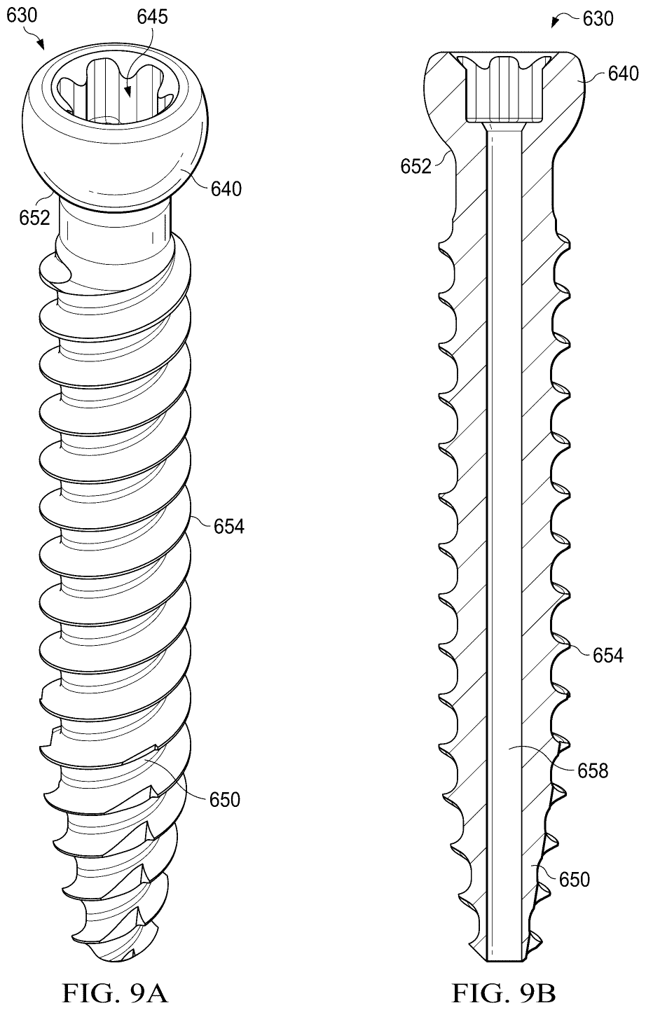

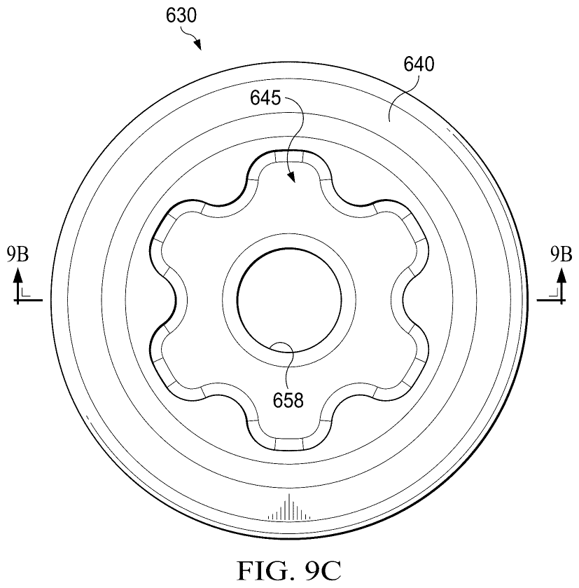

FIGS. 9A-9C depict views of a second bone screw 630. FIG. 9A is a perspective view of a second bone screw 630. FIG. 9B is a cross-sectional view of second bone screw 630. FIG. 9C is a top view of second bone screw 630. In some aspects, second bone screw 630 is similar to bone screw 600 but with a cannula 658. Cannula 658 is an axial bore that defines a lumen through the length of the screw. In the illustrated embodiment, the dual lead pitch screw 630 comprises a head 640 having a spherical-shaped bottom 652 and a shaft 650. Shaft 650 comprises threading 654 that extends completely or partially down the length of the shaft 650. The threading 654 of the dual lead pitch screw comprises two starts. The dual lead pitch screw 630 can be advantageous for use in cortical bone. However, in other embodiments, the screw 630 may not be dual lead and instead can have one start or two or more starts. Moreover, the screw 630 can have any appropriate pitch or lead. Screw 630 includes a drive feature 645 which can be hexalobe shaped as shown in the illustrated embodiment, or can be any other appropriate shape including, but not limited to, hexagonal, square, or triangular.

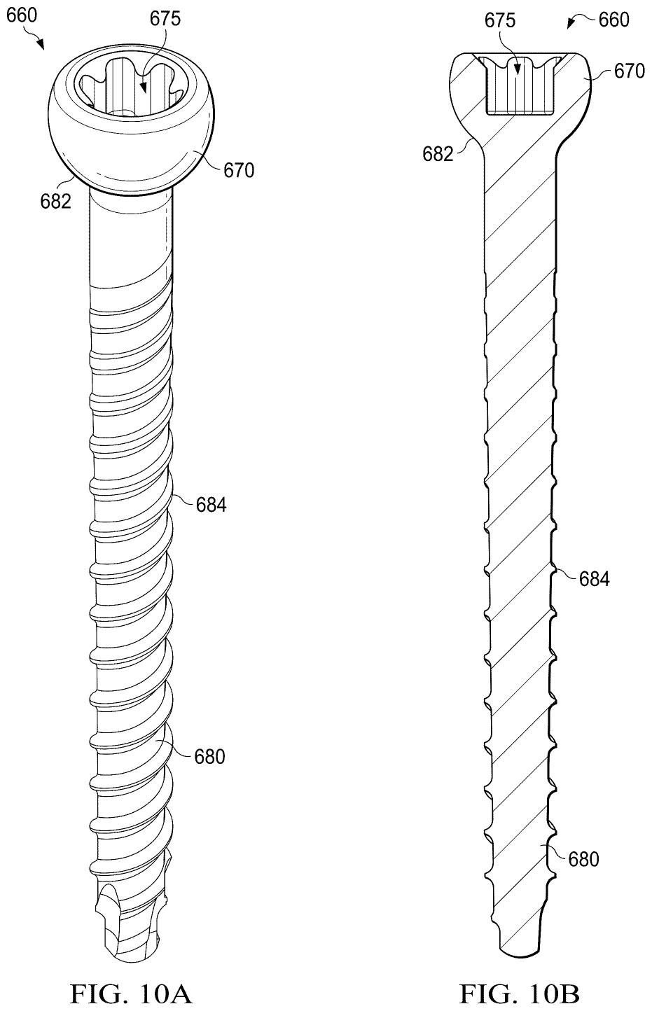

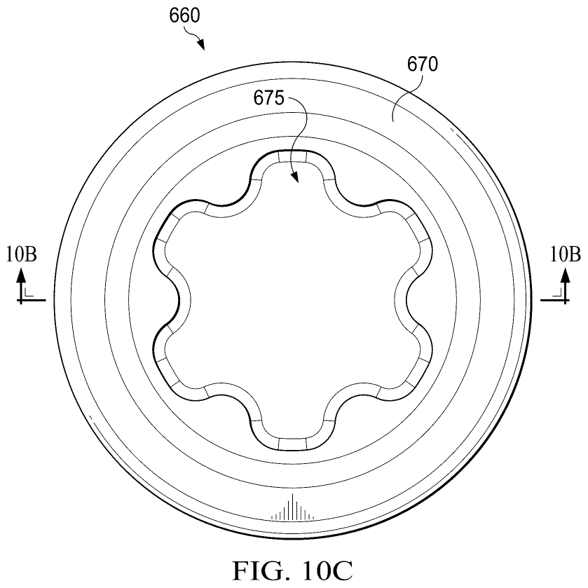

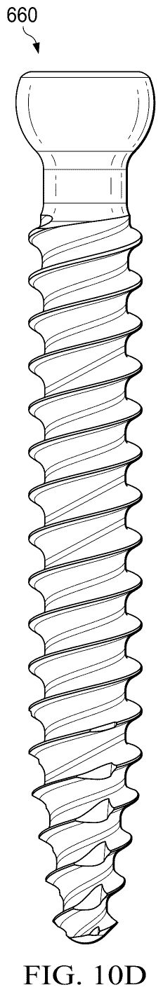

FIGS. 10A-10C depict views of a third bone screw 660 for use in pedicle screw assembly 100. FIG. 10A is a perspective view of a third bone screw 660. FIG. 10B is a cross-sectional view of third bone screw 660. FIG. 10C is a top view of third bone screw 660. In the illustrated embodiment, the dual lead pitch screw 660 comprises a head 670 having a spherical-shaped bottom 682 and a shaft 680. Shaft 680 comprises threading 684 that extends completely or partially down the length of the shaft 680. The threading 684 of the dual lead pitch screw comprises two starts. The dual lead pitch screw 660 can be advantageous for use in cortical bone. However, in other embodiments, the screw 660 may not be dual lead and instead can have one start or two or more starts. Moreover, the screw 660 can have any appropriate pitch or lead. Screw 660 includes a drive feature 675 which can be hexalobe shaped as shown in the illustrated embodiment, or can be any other appropriate shape including, but not limited to, hexagonal, square, or triangular. Screw 660 includes a varying thread length which increases as it goes down the shaft 680 away from the head 670.

FIG. 10D shows another embodiment of the third bone screw 660. The screw 660 in FIG. 10D has some features that are the same as the other bone screws 600, 630, 660 shown and described herein, but includes different cut-outs at its tip. The bone screw in FIG. 10D may be used in place of any of the bone screws described herein.

The receiver 102 of the pedicle screw system 100 can be compatible with any of the screws 600, 630, 660 shown in FIGS. 8A-C, 9A-C, and 10A-D, respectively, or any other appropriate screw according to the embodiments contemplated by the present disclosure. Thus, the same receiver 102 can be used for any appropriate screw 600. This allows the physician to have one type of receiver 102 but choose the desired screw during the procedure. In other aspects, the receiver 102 can be specifically sized, shaped, or otherwise configured for use for one type of screw but not for a different type of screw. The screws can comprise varying shaft thicknesses, as well as other features. Any of the screws described herein can be any appropriate length. For example, the screws can be 20 mm, 25 mm, 30 mm, 35 mm, 40 mm, 45 mm, 50 mm, 55 mm, 60 mm, or any other length. A bone screw can also be referred to as a bone shank, fastener, or other similar term.

FIGS. 11A-11C are cross-sectional views of a receiver 102 at various stages of assembly. FIG. 11A is a cross-sectional view of an unassembled receiver. The unassembled receiver includes the body 200, pressure cap 300, and retainer ring 400 as described in any of FIGS. 1-10D. FIG. 11B is a cross-sectional view of a partially assembled receiver. In FIG. 11A, the pressure cap 300 is introduced into the body 200. The pressure cap 300 is placed in a disengaged position either by inserting through top end 202 or bottom end 204 of the body 200. In the disengaged configuration, protrusions 304 can contact upper side of the detent 220 and/or detent 220 can conform or contact the depression 305 on the outer surface of the pressure cap 300. In other words, an upper portion of the groove 305 can contact an upper portion of the detent 220. As shown, the cross-sectional shapes of the groove 305 and the detent 220 can differ in size and/or shape. For instance, the detent 220 can have a symmetrical cross-sectional shape comprising at least a portion of a semicircle, ellipse, or gaussian curve, for example. The groove 305 can have a non-symmetrical cross-sectional profile. For instance, the non-symmetrical curved profile of the groove 305 can facilitate engagement and disengagement of the pressure cap 300 with the detent 220, while preventing or limiting the ability of the pressure cap 300 to move upward or proximally, whereby the groove 305 would be positioned proximal to the detent 220. In other embodiments, the cross-sectional profile or shape of the groove 305 can be symmetrical. In another embodiment, the cross-sectional profile of the detent 220 can be non-symmetrical.

FIG. 11C is a cross-sectional view of an assembled receiver 102. To complete assembly of the receiver 102, retainer ring 400 is inserted into the body 200 from the bottom end 204 of the body 200. The retainer ring 400 can be split and compressible by a compressing force before being inserted into the body 200. After insertion, the retainer ring 400 rests against the inner conical/tapered surface 232 of the body 200. The lower cavity ledge 236 prevents the retainer ring 400 from falling out of the body 200 once inserted. In other embodiments, no ledge 416 may be provided, such that the conical or tapered shape of the surface 232 itself prevents the retainer ring 400 from falling out of the body 200.

In some aspects, the assembly shown in FIGS. 11A-11C can occur at a manufacturing facility or assembly facility. The assembled receiver 102 of FIG. 11C can be packaged in a sterile package for delivery to hospitals, clinics, physicians, and/or medical device distribution centers. The packaged receiver assemblies 102 can accompany packaged bone screws 600. Accordingly, a packaged receiver 102 and a packaged bone screw 600 can be selected by a physician prior to or during a spinal fixation procedure (or any other relevant procedure) based on the patient's anatomy and the specifics of the surgical routine. As explained below, a further assembly procedure is described in FIGS. 12A-12D, whereby a bone screw 600 is inserted into the assembly, the pressure cap 300 is engaged to provide a friction fit, and the set screw is inserted and tightened against the rod. In some aspects, the assembly shown in FIGS. 12A-12D can be performed at the time and place of surgery. In other aspects, however, the assembly shown in FIGS. 12A-12D can be performed in conjunction with the assembly of FIGS. 11A-11C, for instance, in a manufacturing facility.

FIGS. 12A-12D depict cross-sectional views of a pedicle screw assembly 100 at various stages of assembly. The components depicted in FIGS. 12A-12D can comprise any of the embodiments described in FIGS. 1-11C. FIG. 12A is a cross-sectional view of an unassembled pedicle screw assembly. The unassembled pedicle screw assembly, a receiver 102 and bone screw/shank 600. FIG. 12B is a cross-sectional view of a partially assembled pedicle screw assembly 100. FIG. 12A illustrates inserting the bone screw/shank 600 through the bottom end 204 of the receiver 102. During the insertion of the bone screw 600, the retainer ring 400 can translate longitudinally away from the bottom end of the 200. Once the bone screw 600 is inserted an appropriate distance, the retainer ring 400 can come to rest around the screw/shank head 610, where the inner surface 415 of the retainer ring 400 is in contact with the screw head 610 and prevents the screw from falling out of the receiver 102. FIG. 12C is a cross-sectional view of a partially assembled pedicle screw assembly. When the pressure cap is pressed longitudinally towards the distal end of the receiver 102, the protrusions 304 snap past the detent 220 and the inner surface 314 of the pressure cap 300 comes in contact with the screw head 610. FIG. 12D is a cross-sectional view of an assembled pedicle screw assembly 100. Finally, the rod 120 can be inserted, and aligned with the U-shaped slot 214. Set screw 500 can be screwed into the body 200 until it compresses the rod 120, which thereby compresses the pressure cap 300, which thereby compressed the screw head 610, locking the orientation of the screw 600.

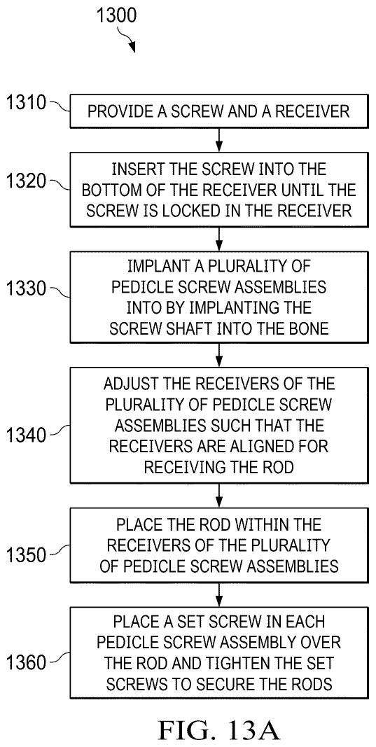

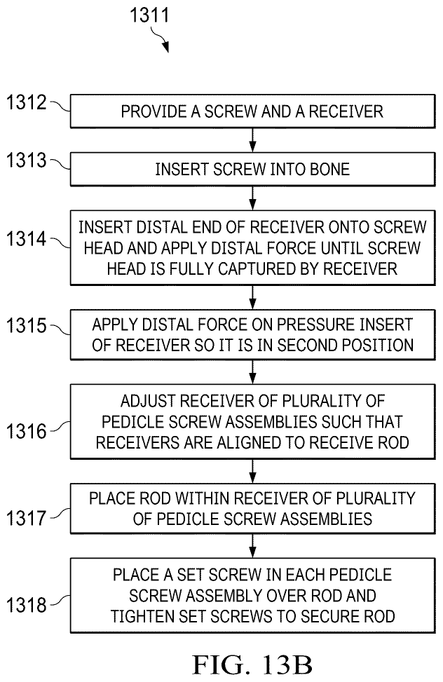

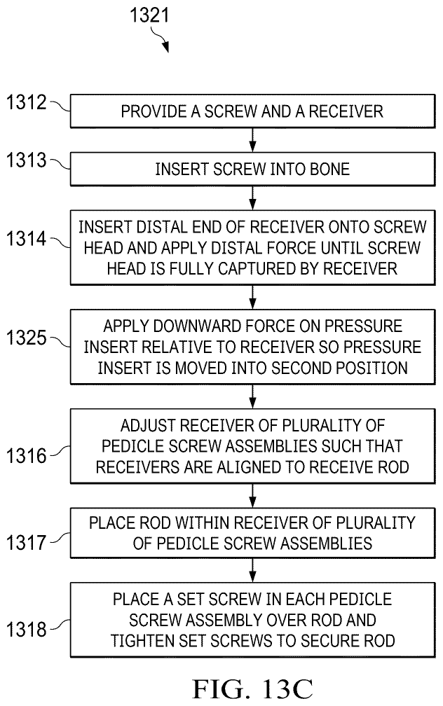

FIG. 13A shows a method 1300 of preparing the pedicle screw assembly 100 for use to subsequently treat a spinal condition using the pedicle screw assembly 100. Step 1310 of the method 1300 includes providing a screw 600 and a receiver 102. The receiver 102 can be any receiver 102 described herein, including any of the receivers 102 or parts of receivers 102 shown in any of the FIGURES herein. This includes without limitation, FIGS. 1-3, 4A-4C, 5A-C, 6A-6D, 7A-7C, 8A-8C, 9A-9C, 10A-10D, 11A-11C, and 12A-12D. The screw 600 can be any screw 600 described herein, including the screws 600 shown in FIGS. 1, 8A-8C, 9A-9C, and 10A-10D. The surgeon or technician can select a receiver 102 and fastener assembly from a plurality of receiver 102 and fastener assemblies based on the patient's anatomy and/or indications. The modularity of the components allow them to be used in any of a variety of styles and sizes of pedicle screw assemblies to meet the patient's anatomy and/or indications. Step 1310 can incorporate aspects of FIGS. 11A-11C and/or of FIG. 12A.

Step 1320 of the method 1300 includes inserting the screw 600 into the bottom 204 of the receiver 102 until the screw 600 is locked in the receiver 102, thereby forming a pedicle screw assembly 100. FIG. 12A shows the screw 600 prior to insertion into the bottom 204 of the receiver. As the screw 600 moves upward through the receiver 102, the retainer ring 400 can expand over the head 610 of the screw 600, to a position shown in FIG. 12B, where the screw head 610 has passed through the retainer ring 400. When the upward force is removed from the screw 600, the screw 600 can move downward slightly to rest in the base 212 of the body 200, as shown in FIG. 12B. As the screw 600 moves downward, the retainer ring 400 can move downward. A force can be applied through the top end 202 of the body 200 to move the pressure cap 300 from the disengaged position (a first position) to the engaged position (a second position) as described herein, and as shown in FIG. 12C. However, the screw 600 can be inserted into and be locked in the receiver 102 in any way as described herein. Step 1320 can incorporate aspects of FIGS. 12A-12C, for instance.

Step 1330 of the method 1300 includes implanting a plurality of pedicle screw assemblies 100 into bone by implanting the screw shaft 620 into the bone. In some embodiments, the bone can be a vertebra 110 (FIG. 1). As described herein, an instrument can releasably engage the engagement feature 206 of the receiver 102. The instrument can then be used to position the pedicle screw assembly 100 at the desired position. The same instrument or a different instrument can drive the screw 600 into the bone. For example, a screw driver can pass through the opening 302 of the pressure cap 300 to access the screw head 610. The screw driver can then be used to drive the screw shaft 620 into the bone. However, the screw 600 can be driven into the bone using any appropriate method. Step 1340 of the method 1300 includes adjusting the receivers 102 of the plurality of pedicle screws 100 such that the receivers 102 are aligned for receiving a rod 120. An instrument can engage with the engagement feature 206 of the receiver 102 to move the receiver 102 independent of the screw 600. The receiver 102 can be moved by a user's hand or manual instrument into any appropriate position for receiving a rod 120. The frictional force that the pressure cap 300 applies to the screw head 610 can retain the position and orientation of the receiver 102 relative to the screw head 610. In this way, the receiver 120 may be individually adjusted and aligned for receiving a rod without flopping due to the force of gravity or other low inadvertent forces.

Step 1350 of the method 1300 includes placing a rod 120 within the receivers 102 of the plurality of pedicle screw assemblies 100. Once the receivers 102 are aligned, a rod 120 can be placed such that it fits within the U-shaped slot 214 formed by the arms 210 of the receiver 102. The rod 120 can be bent or curved into the desired shape before or while placing the rod 120 into the receiver 102. Step 1360 of the method 1300 includes placing a set screw 500 in each pedicle screw assembly 100 over the rod 120 and tightening the set screws 500 to secure the rod 120. Tightening the set screws 500 can also secure the position and orientation of the receivers 102 relative to the screws 600. The set screws 500 can be any appropriate set screw 500 design, including the design shown in FIGS. 1 and 7A-7C. The set screw 500 can have threads that engage the threads 216 of the arms 210 of the body 200. An instrument such as, for example, a screwdriver, can be used to tighten the set screw 500 until it contacts and presses against the rod 120. The set screws 500 can secure the rod 120 and/or the receivers 102 such that they do not move. In some embodiments, the rod 120 can be tightened such that it stabilizes the vertebrae 110. Steps 1350 and 1360 can incorporate aspects of FIG. 12D, for instance.

FIG. 13B provides a method 1311 of assembling a spinal screw assembly. This example method can be used to assemble any of the screw assemblies described herein. At a step 1312, the method includes providing a screw and receiver, such as the screw 600 and the receiver 102. This step is also described as step 1310 in the method 1300 (FIG. 13A).

At step 1313, a user may insert the screw 600 into bone. This may be done using known methods using either powered or manual force to engage the head 610 of the screw 600. With the screw 600 stabilized in the bone so that the head 610 and the proximal portion of the shank project out of the bone, the distal end of the receiver may be inserted onto the screw head, and distal force may be applied until the screw head is fully captured by the receiver, as in step 1314. Step 1314 is consistent with the description herein relating to step 1320 in the method 1300 and the discussion relating to FIGS. 12B and 12C.

At step 1315, a user may apply a distal force on the pressure insert 300 of the receiver so it is in the second position or engaged position. Details of step 1315 are described herein with reference to FIGS. 12B and 12C. In so doing, the pressure insert 300 moves from the disengaged position (such as a first position shown in FIG. 12B) to the engaged position (such as the second position) shown in FIG. 12C. As a part of this transition from the disengaged position to the engaged position, the pressure cap moves longitudinally towards the distal end of the receiver 102, the protrusions 304 snap past the detent 220, and the inner surface 314 of the pressure cap 300 comes in contact with the screw head 610.

When the pressure insert 300 is disengaged from the head 610 of the screw 600 (the first position), the receiver may flop or rotate subject to gravitational or such minimal force so as to not hold the receiver 200 in place relative to the head. However, when the pressure insert 300 is engaged with the head 610 in the engaged position (the second position), the pressure insert 300 applies sufficient force on the head 610 to hold the receiver in a desired position, such as a pre-lock position. This pre-lock position still permits a user to manually orient the receiver in a desired position (such as a position to receive the rod), and the friction due to the engagement holds the receiver in the desired orientation (such that it does not flop over on its own). Furthermore, as indicated herein, the detent 220 interferes with the protrusion 304 so that the pressure cap cannot return to the first position without the application of a sufficient overcoming force. This maintains the inner surface 314 of the pressure cap 300 in contact with the screw head 610 to provide the pre-lock friction force.

At a step 1316, the user may adjust the receiver of a plurality of pedicle screw assemblies such that the receivers are aligned to receive the rod. This may include both rotating and pivoting the receivers 200 with respect to their respective screws. Since the pedicle screw assembly is in the engaged position (second position), the frictional engagement of the screw head with the pressure insert (and the retainer ring 400) holds the receiver in place relative to the screw so that it does not freely move without the application of a force.

At a step 1317, the rod is placed within the receivers of the plurality of pedicle screw assemblies. At a step 1318, a set screw is placed in each pedicle screw assembly over the rod and the set screws are tightened to secure the rod, as shown in FIG. 12D.

FIG. 13C provides another method 1321 of assembling a spinal screw assembly. This example method can be used to assemble any of the screw assemblies described herein. Method 13C has the same method steps as those described with reference to the method 1311 in FIG. 13B, except for step 1325. At step 1325, the method 1321 includes a step of applying a downward force on the pressure insert relative to the receiver so the pressure insert is moved into the second position. As noted above, the second position is where the pressure insert is engaged against the screw head 610 so as to create sufficient friction to pre-lock or hold the receiver in place relative to the screw so as to not slip or fall due to small forces, such as gravity, but that still permits a user to manually adjust the receiver relative to the screw to a desired position, and the friction force holds the receiver in the desired position.

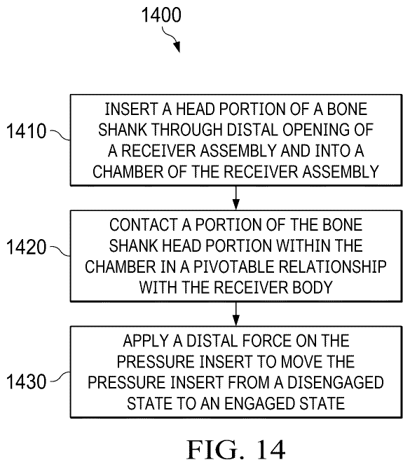

FIG. 14 shows a method 1400 of assembling a spinal screw assembly. It will be understood that the method 1400 can be used to assemble any of the screw assemblies described herein, including those illustrated in FIGS. 1-12D. At step 1410, the physician inserts a head portion of a bone shank through a distal opening of a receiver assembly and into a chamber of the receiver assembly. The receiver assembly comprises a receiver body, comprising: the distal opening; a conical interior surface disposed about the chamber; an axial bore extending longitudinally through the receiver body; and a detent defined in a surface of the axial bore. The receiver further comprises a split retainer ring disposed in the chamber; and a pressure insert disposed at least partially within the axial bore. The pressure insert comprises a circumferential depression and/or protrusions on an exterior surface of the pressure insert.

At step 1420, by inserting the head portion of the bone shank through the distal opening, the head portion pushes through the distal opening of the receiver body and through the split retainer ring to cause the split retainer ring to elastically expand about the head portion, and thereafter to contact a portion of the bone shank head portion within the chamber in a pivotable relationship with the receiver body.

At step 1430, the physician applies a distal force on the pressure insert to move the pressure insert from a disengaged state to an engaged state as described herein. In the engaged state, the protrusion contacts a detent of the receiver from the bottom of the bottom detent as described herein. It will be understood that the method 1400 can incorporate aspects of FIGS. 12A-12D.

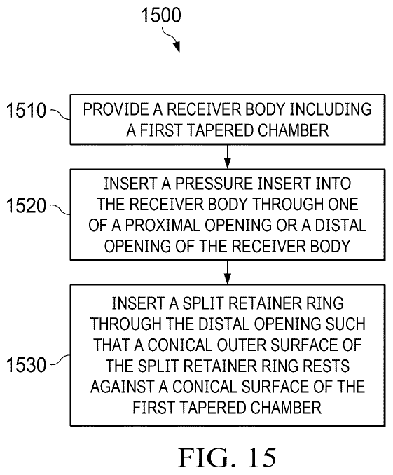

FIG. 15 shows a method 1500 of assembling a receiver assembly. It will be understood that the method 1500 can be used to assemble any of the receiver assemblies described herein, including those illustrated in FIGS. 1-12D. At step 1510, a technician or assembler provides a receiver body. The receiver body comprises: an axial bore extending longitudinally through the receiver body from a proximal opening of the receiver body to a distal opening, the distal opening comprising a width; a first tapered chamber disposed adjacent to the distal opening, wherein the first tapered chamber increases in diameter toward a top portion of the first tapered chamber.

At step 1520, the assembler inserts a pressure insert into the receiver body through one of the proximal opening or the distal opening. Inserting the pressure insert can include engaging at least one resilient tab of the pressure insert past a detent of the receiver body. As explained above, in an exemplary embodiment, the pressure insert includes protrusions on opposing sides of the pressure insert and a circumferential depression.

At step 1530, the assembler inserts a split retainer ring through the distal opening such that a conical outer surface of the split retainer ring rests against a conical surface of the first tapered chamber. In some aspects, step 1530 comprises compressing the split retainer ring from a first width to a second width, wherein the second width of the split retainer ring is smaller than the width of the distal opening.

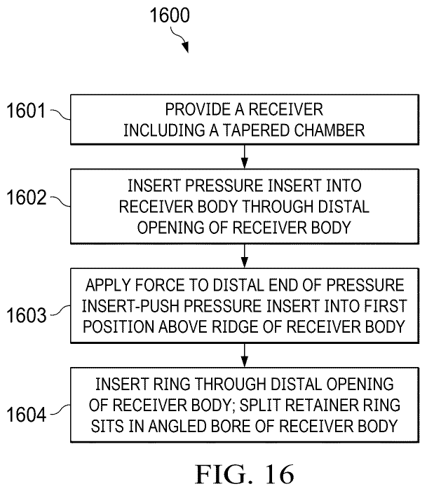

FIG. 16 shows an example method 1600 of assembling a pressure insert 300 and a retainer ring 400 or 440 into an example receiver body. In step 1601, a user provides a receiver that includes a tapered chamber therein. One example of such a receiver is shown in FIG. 4A, with the surface 232 having a conical shape or a taper. In step 1602, the user inserts the pressure insert 300 into the body 200 of the receiver through the distal opening at the bottom end 204 of the receiver body 200 (in FIG. 4A). The pressure insert 300 may have a diameter smaller than the distal opening at the bottom end of the receiver. In step 1603, the user applies force to the distal end of the pressure insert to push the pressure insert into a first position (such as the upper or disengaged position). In this first position the pressure insert then resides at least partially above the internal ridge (or bumps) of the receiver body. In so doing, in some implementations, a portion of the pressure insert, such as the upper portion of the pressure insert elastically deforms as it slides past the internal ridge of the receiver body. Once past the internal ridge, the pressure insert may elastically return to its shape with the internal ridge of the receiver in the circumferential depression. This secures the pressure insert in the receiver body in the first position.

In step 1604, the user subsequently inserts the retainer ring through the distal opening of the receiver body, whereby the retainer ring (e.g., a split retainer ring) then sits in the angled bore or tapered chamber of the receiver body. Since the retainer ring may have a diameter smaller than the diameter of the distal opening at the bottom end of the receiver, this step may include elastically deforming the retainer ring to have a smaller diameter, introducing the retainer ring through the distal opening, and allowing the retainer ring to snap back toward its original neutral diameter. In this way, the retainer ring may be disposed within the angled bore of the receiver body.

The various components of the implantable receivers 102 are sized and shaped to modularly cooperate to permit customized pedicle screw assemblies 100 to meet particular needs. For example, in some implementations, the receiver body 200 may be sized to receive any of a variety of types, styles, and sizes of screws, so long as the screws share the same size screw head. Likewise, different varieties of pressure inserts may have a common size and shape to cooperate with the receiver body, although they have may different saddle shapes. This allows for modular assembly before or during spinal fixation, enabling bottom-side loading of the screw head into the receiver. Various screws with different characteristics can be coupled to the receiver body before or after implantation. This sort of modular approach may enable reduced inventory of specific parts, reducing overall costs and reducing the number of components to be kept on hand to meet needs.

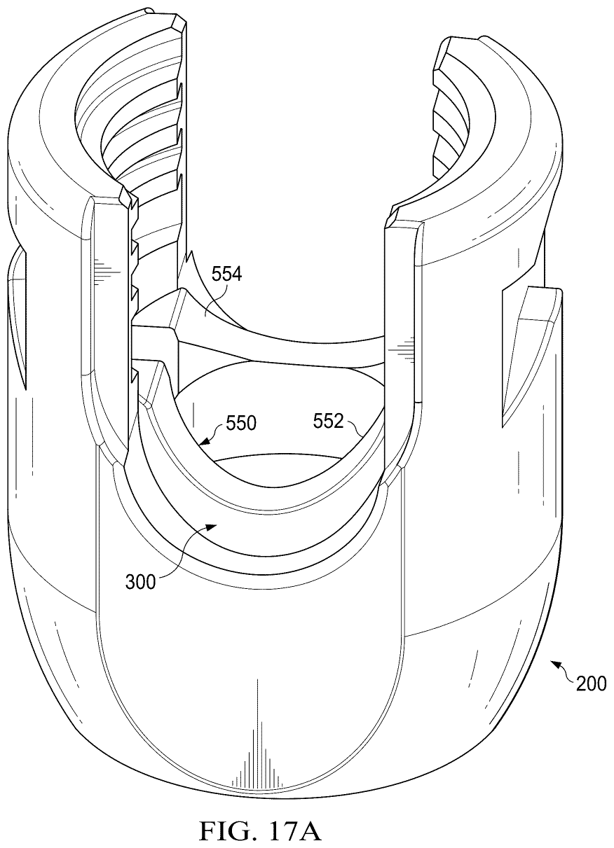

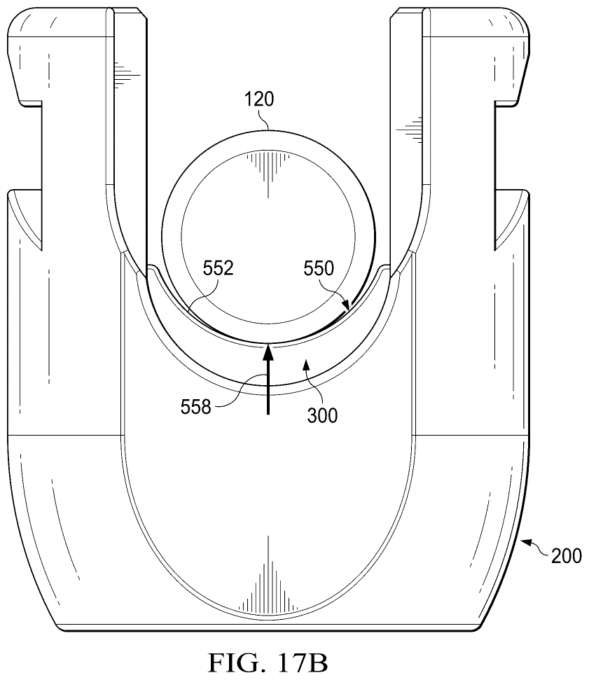

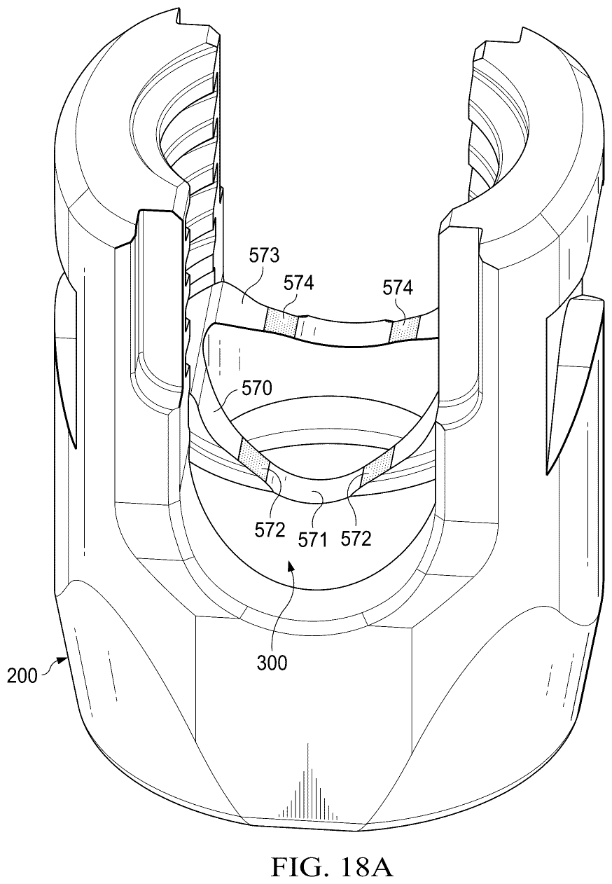

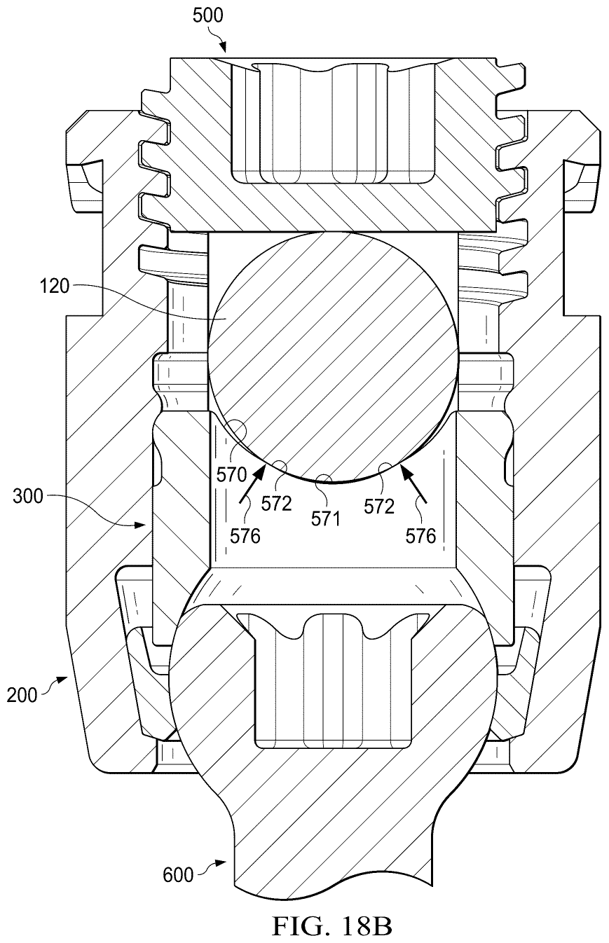

FIGS. 17A-17B and 18A-18B show example saddle shapes that may form a part of any of the embodiments described herein. In an embodiment shown in FIG. 17A, an example saddle 550, which may form a part of the pressure insert 300, may be disposed in a tulip or receiver body 200. The saddle 550 in this example is shaped as a continuous arc that receives and affixes a fixation rod. In this embodiment, FIG. 17A shows the front portion 552 and the rear portion 554 of the saddle 550 and the corresponding continuous arc of each portion of the saddle 550 prior to rod installation into the tulip or receiver body 200.

In FIG. 17B, a fixation rod 120 installed in the tulip contacts the continuous arc of the saddle 550 at a single contact point on the front portion 552 of the saddle (the arrow 558 indicates the contact point in FIG. 17B), and the fixation rod 120 simultaneously contacts the continuous arc at a corresponding single point on the rear portion 554 of the saddle (not shown in FIG. 17B). In one or more embodiments, the fixation rod 120 can be a shape that is generally round or elliptical.

In an embodiment shown in FIG. 18A, another example saddle 570 installed in a tulip or receiver body 200 includes a front portion 571 and a rear portion 573 that each provides a partial arc. The front portion 571 includes flat cutouts 572 identified by dark colored sections on the front portion 571 of the saddle 570, and the rear portion 573 includes flat cutouts 574 on the rear portion 573 of the saddle 570. In one or more embodiments, the flat cutouts are generally angled relative to each other so as to form portions of a V-shape for receiving a corresponding fixation rod. In this embodiment, the portions of the V-shape provide a line contact at two points on both the left and the right sides of the saddle 570, resulting in four points of line contact—one along each of the flat cutouts. In an embodiment, the flat cutouts are sized and spaced apart from each other to form four points of contact along a fixation rod having particular sizes, such as for example, fixation rods with 5.5 mm and 6.0 mm diameter rods. Other fixation rod diameters can also be used. In addition, in some embodiments, the saddle is V-shaped. The fixation rod engages the four points of contact as described herein, providing consistent and predictable gripping performance between the rod and the saddle. In one or more embodiments, the fixation rod can be a shape that is generally round or elliptical.

FIG. 18B illustrates a fixation rod 120 installed in the saddle 570 and in the receiver body. FIG. 18B shows a partial cross-section taken through all components except the pressure insert. The fixation rod 120 contacts the flat cutouts 572 shown in FIG. 18A at two points on the front portion 571 of the saddle (the arrows 576 indicate the contact points in FIG. 18B). In addition, the fixation rod 120 simultaneously contacts the flat cutouts 574 at two corresponding points on the rear portion 573 of the saddle (not shown in FIG. 18B, but the flats can be seen in FIG. 18A). In the example in FIG. 18B, the installed set screw 500 has driven the top of the fixation rod 120 against the pressure insert 300 and forced the rod 120 to seat at the indicated flat cutouts 572, 574 of the saddle 570.

Aspects, components, and features described above can be used in a variety of skeletal stabilization and/or fixation systems. For example, although the pressure cap described above is shown in low-profile, singular receivers, the present disclosure contemplates other types of receivers and spinal implant devices. For example, the pressure cap can be incorporated into reduction screw receiver bodies, sliding double bodies, closed receiver bodies, and/or any other suitable type of spinal implant or receiver body. Further, although embodiments of the present disclosure may be described as spinal implants or spinal fixation devices, it will be understood that the devices described above can be used for a variety of skeletal stabilization and/or fixation procedures.

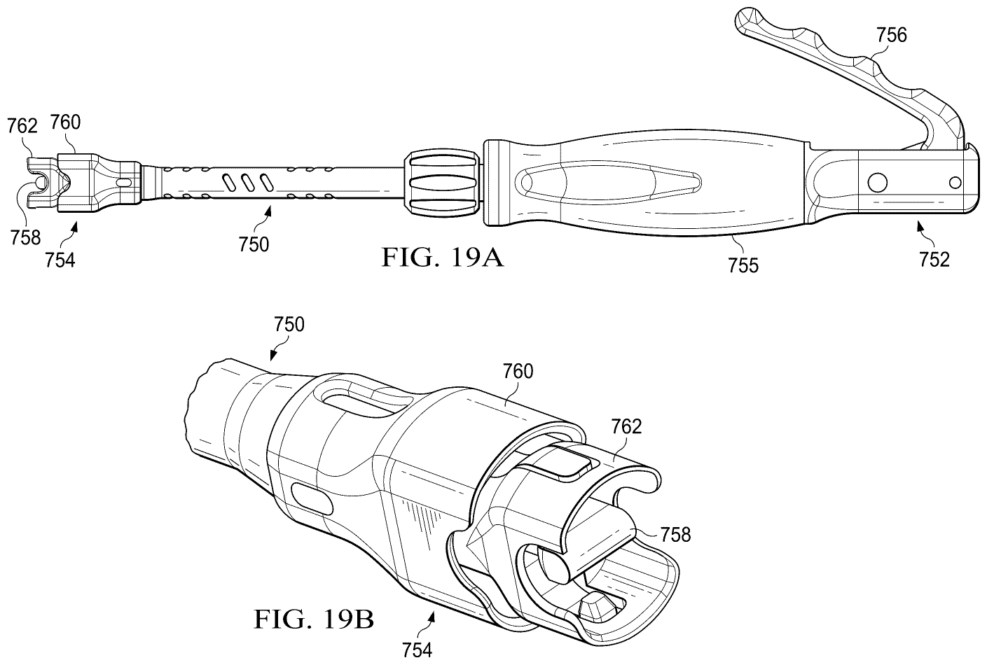

FIG. 19A illustrates an example embodiment of a tulip inserter (also referred to as an inserter tool) 750. The tulip inserter 750 includes a proximal end 752 and a distal end 754. The proximal end 752 includes a grippable portion 755 and a lever arm 756. An inner plunger 758 extends internally in the tulip inserter 750 from the distal end 754 toward the proximal end 752. A distal engagement housing 760 houses a distal portion of the plunger 758.

FIG. 19B illustrates an example embodiment of the distal end 754 of the tulip inserter inner plunger 758 in an expanded perspective view in relation to FIG. 19A. The distal end 754 includes spring arms 762 configured to engage with a receiver body, such as the receiver body 200 described herein.

The tulip inserter 750 is configured to engage with the receiver body 200 and, use the inner plunger 758 to apply a load against the pressure insert 300 to press the pressure insert 300 so that the pressure insert moves or displaces from the first or disengaged position to the second or engaged position. FIG. 19C illustrates an example embodiment of a tulip inserter distal engagement housing 760 in a partial cross-section view. In this example, the pressure insert 300 is shown in a side view. As shown in FIG. 19C, distal engagement housing 760 of the tulip inserter 750 is shown with spring arms 762 engaged with a tulip or with the outside surfaces of the receiver body 200. The spring arms 762 provide passive retention of the distal engagement housing 760 of the inserter 750 with the receiver body. The inner plunger can displace relative to the spring arms 762 to engage the saddle 764 of the pressure insert 300 and displace the pressure insert from the first position or disengaged position toward the second position or the engaged position.

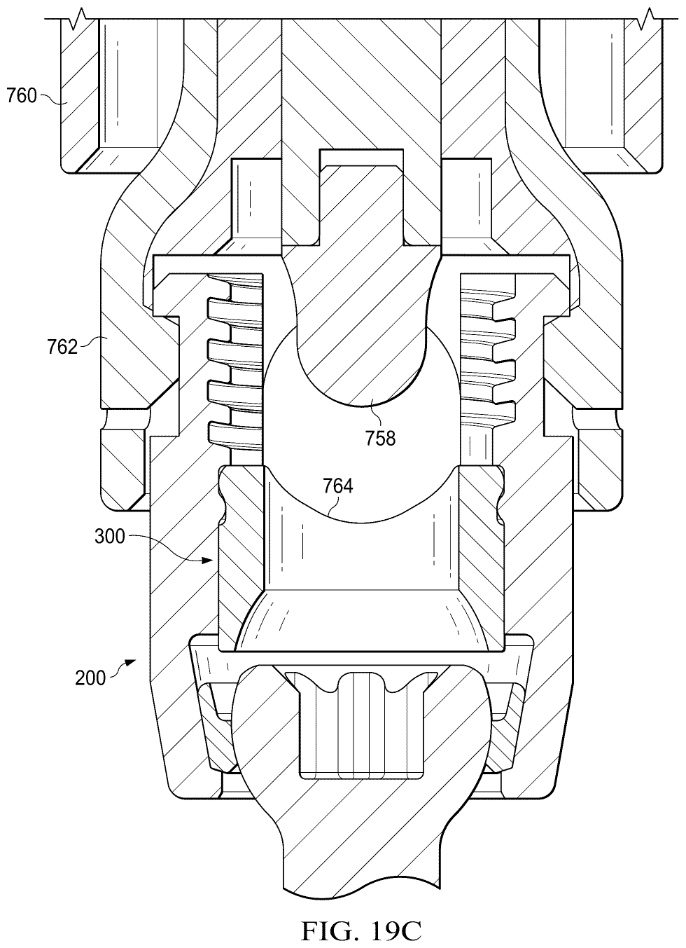

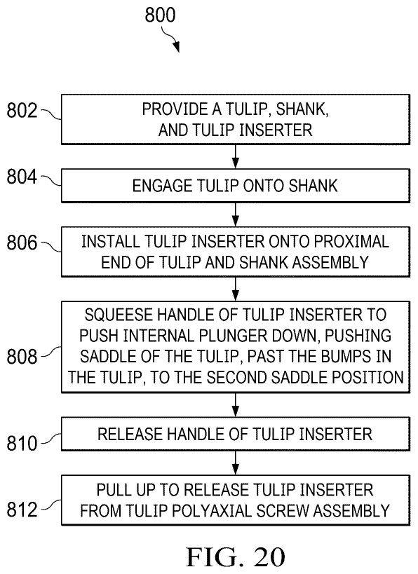

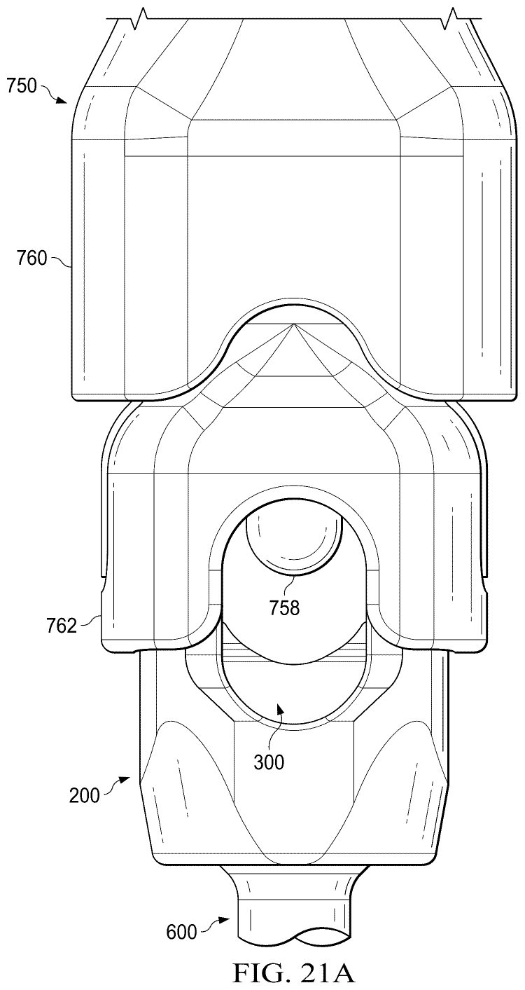

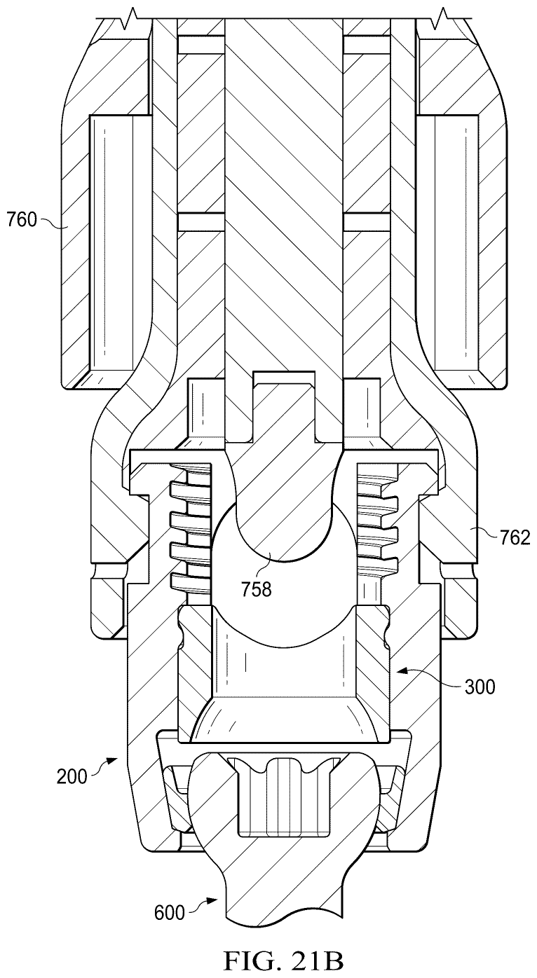

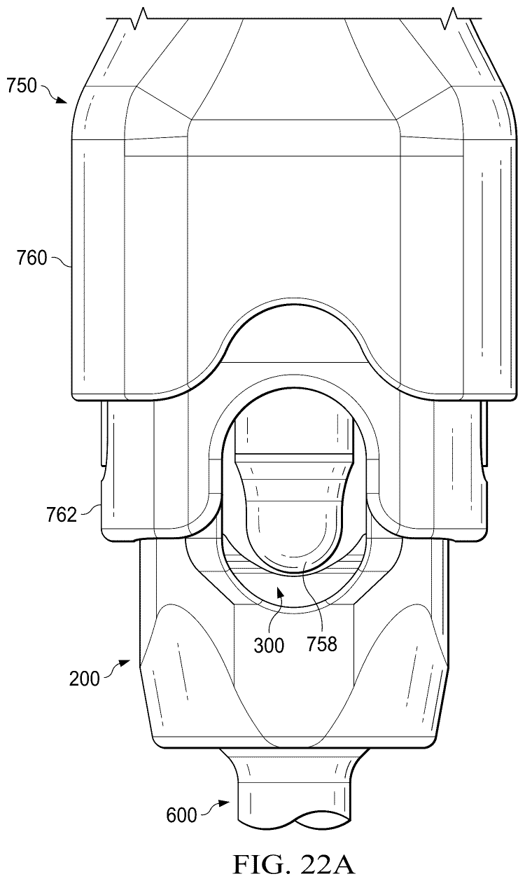

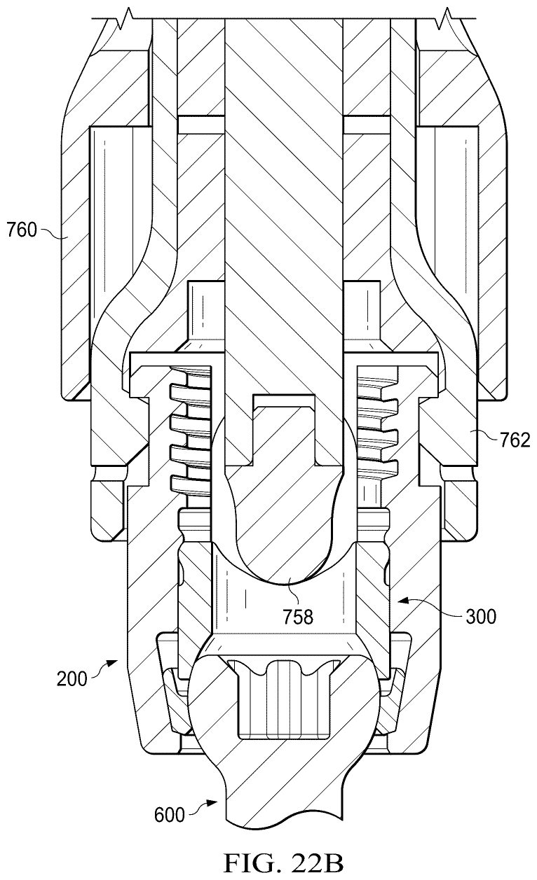

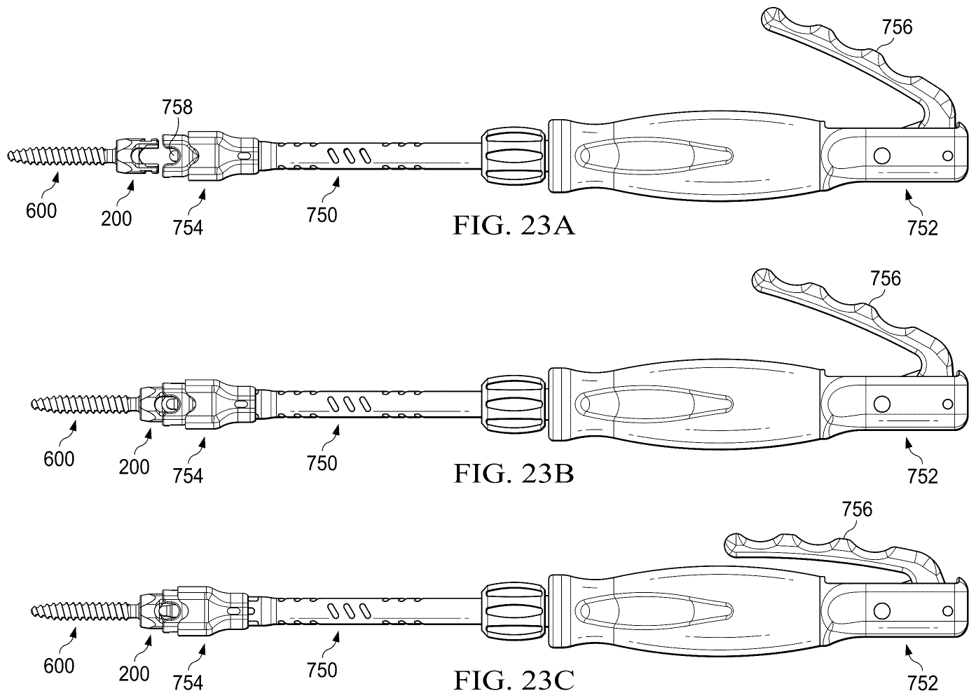

FIG. 20 shows a method 800 of using a tulip inserter 750 to push or move the pressure insert 300 from a first or disengaged position to a second or engaged position on a head of the bone screw 600. This method is described with reference FIGS. 21A, 21B, 22A, 22B, and 23A, 23B, and 23C. FIGS. 21A and 21B and FIGS. 22A and 22B show the tulip inserter 750 being used to displace the pressure insert 300. FIGS. 23A, 23B, and 23C show the tulip inserter 750 engaging with a screw assembly and show displacement of the lever to move the plunger.

With reference to FIG. 20, the method 800 begins at a step 802 with providing a tulip or receiver body 200, a shank or screw 600, and the tulip inserter 750. At step 804, a user engages a tulip onto the screw shank 600 that is already installed in a target bone site, such as a vertebra. In some examples, the tulip was engaged onto the screw shank using any of the methods described herein, including without limitation, the method steps 1312, 1313, and 1314 from the method of FIG. 13B. This step is also shown in FIG. 23A, although without showing the bone.

At step 806, the user then engages the inserter tool 750 onto the proximal end of the tulip assembly or receiver body 200. This can include engaging the spring arms 762 of the distal engagement housing 760 of the inserter tool 750 with the upper arm portions of the tulip or body 200. Such a position is shown in FIGS. 21A and 21B, and shown more generally in FIG. 23B. With reference to FIGS. 21A and 21B and 23B, the spring arms of the inserter tool are engaged against the outer surface of the tulip or receiver body 200. The inner plunger is spaced apart from the pressure insert 300, and the pressure insert is shown in the first position disengaged from the screw 600. Since the pressure insert is shown in the first position disengaged from the screw 600, receiver is not configured to remain in its position relative to the screw 600. Instead, it is loosely fitted and can be subject to moving when not subject to an applied force. That is, it can flop or fall.

At step 808, the user then squeezes the handle of the inserter tool 750 to force the inner plunger 758 distally against the proximal portion of the pressure insert (i.e., the saddle) within the tulip body 200, and the inner plunger 758 correspondingly drives the pressure insert 300 in a distal direction within the tulip chamber. This applied load causes the pressure insert 300 to progress over and past the internal bumps, ridges, or detents of the receiver body (shown as detent 220 in FIG. 12D) of the tulip chamber, so that the pressure insert 300 rests in the second or engaged position. The second position or engaged position is shown in FIGS. 22A and 22B and 23C. In these FIGURES, the tip of the plunger 758 has engaged the saddle and driven the pressure insert 300 downward relative to the tulip or receiver body 200 to the second or engaged position, where the head 610 of the shank or screw 600 is engaged between the pressure cap 300 and the retainer ring 400 (or 440).

At step 810, the user releases pressure on the inserter tool lever arm 756. This allows the inner plunger 758 to retract into the inserter tool 750. At the step 812, the user further pulls up or longitudinally displaces the inserter tool 750 relative to the tulip assembly to release the inserter tool 750 from the tulip assembly.

An advantage of the friction-fit design described herein is the various stages obtained during the assembly or implantation process. At a first stage or free stage, the receiver body may contain the pressure insert and the retainer ring. The screw may be fully unattached from the receiver body. At this stage, the screw is in a free state because it is not retained in the receiver body. Also at this stage, the provider may choose a desired style and size of screw from a plurality of screws having different styles and sizes. Here, the receiver is not limited by the screw at all.

At a second stage or loosely engaged stage, the screw is introduced into the receiver body. As described herein, the screw head passes through the distal or bottom end of the receiver. Since its size is greater than the inner diameter of the retainer ring, the retainer ring elastically expands and the head of the screw passes through the retainer ring. At this stage, the screw and the receiver are connected, and the receiver may loosely pivot or rotate about the head of the screw. Thus, in some instances of the second stage, the receiver may be loose enough on the head of the screw to pivot freely even as a result of gravity. This may allow the receiver body to flop about the screw head.

At a third stage or pre-locked stage, the pressure insert has been displaced from its first position or disengaged position to a second position or engaged position. In this stage, the pressure insert is applying some loading against the screw head in a manner that increases friction sufficiently to permit a user to manipulate the receiver body relative the screw head and the friction between the pressure insert and the screw head and the friction between the retainer ring and the screw head maintains the position of the receiver body relative to the screw head. At this stage, the receiver body will not flop due to gravity but instead maintains any desired position of the user. Since the third stage is not a final lock stage, the receiver body can still be manually manipulated, using a hand or an inserter tool, to a desired position. In some instances, the desired position is one where the receiver body is aligned or oriented to a position to receive the spinal rod. Being able to align the receiver bodies prior to introducing the spinal rod can simplify and save time when implanting the spinal rod on the patient.

At a fourth stage or locked stage, the surgeon has introduced a spinal rod into the aligned receiver bodies, and the set screw is used to apply loading against the spinal rod. The spinal rod in turn applies loading against the pressure insert, thereby clamping the screw head between the pressure insert and the retainer ring. The system is tight so as to prevent displacement of the receiver body relative to the screw head. This is a fully locked or secure stage and is the final stage securing the spinal rod to the patient in a manner preventing movement of the spine. With the pedicle screw assembly in the fourth stage, the spinal stabilization is complete.

Persons of ordinary skill in the art will appreciate that the implementations encompassed by the present disclosure are not limited to the particular exemplary implementations described above. In that regard, although illustrative implementations have been shown and described, a wide range of modification, change, combination, and substitution is contemplated in the foregoing disclosure. It is understood that such variations can be made to the foregoing without departing from the scope of the present disclosure. Accordingly, it is appropriate that the appended claims be construed broadly and in a manner consistent with the present disclosure.

1. A fastener assembly for a spinal fixation system, comprising:
a bone shank comprising a head portion and a distal threaded portion configured to be implanted into bone;
a receiver comprising:
a channel for receiving a fixation rod therein;
an axial bore extending longitudinally through the receiver from a proximal opening of the receiver to a distal opening, the distal opening being sized and shaped to receive the head portion of the bone shank therethrough;
a chamber disposed adjacent to the distal opening, the chamber comprising a conical interior surface, the conical interior surface comprising a distal portion having a first diameter, and a proximal portion having a second diameter larger than the first diameter; and
a ridge protruding from a surface of the axial bore;
a split retainer ring disposed in the chamber, the split retainer ring being configured to expand within the chamber to allow the head portion of the bone shank to pass therethrough and thereafter support the head portion in a pivotable relationship; and
a pressure insert disposed at least partly above the split retainer ring, the pressure insert comprising:
a saddle configured to seat the fixation rod;
a distally-facing concave surface configured to contact the head portion of the bone shank;
a depression formed in an exterior surface of the pressure insert; and
a rounded corner adjacent to a proximal end of the pressure insert,
wherein, when the pressure insert is in a first longitudinal position relative to the receiver, the depression is configured to engage the ridge in the receiver to maintain the pressure insert at the first longitudinal position; and
wherein, when the pressure insert is in a second longitudinal position distal, the ridge is disengaged from the depression and urged against the rounded corner of the pressure insert such that the head portion of the bone shank is maintained in a friction fit with both the pressure insert and the split retainer ring.

2. The fastener assembly of claim 1, wherein the head portion of the bone shank comprises a spherical surface, wherein the split retainer ring comprises an interior concave surface configured to contact the spherical surface to thereby provide the pivotable relationship.

3. The fastener assembly of claim 2, wherein:
the receiver comprises an interior conical surface defining the chamber,
the split retainer ring further comprises an outer conical surface, and
when the pressure insert is in the second longitudinal position, the outer conical surface abuts the interior conical surface of the receiver.

4. The fastener assembly of claim 3, wherein the split retainer ring comprises a groove formed in a proximal end of the split retainer ring, wherein a distal end of the pressure insert is seated in the groove when the pressure insert is in the second longitudinal position.

5. The fastener assembly of claim 4, further comprising a compression screw configured to be threadably received into an upper opening of the receiver along a longitudinal axis of the receiver, and wherein the compression screw is configured to compress the fixation rod against the pressure insert, which in turn locks the fastener assembly by simultaneously urging:
the spherical surface of the head portion of the bone shank against the interior concave surface of the split retainer ring; and
the outer conical surface of the split retainer ring against the interior conical surface of the receiver.

6. The fastener assembly of claim 1, wherein the depression comprises a groove extending circumferentially across the exterior surface of the pressure insert.

7. The fastener assembly of claim 6, wherein the groove comprises a non-symmetrical cross-sectional profile.

8. The fastener assembly of claim 7, wherein the ridge comprises a symmetrical cross-sectional profile.

9. A receiver for a polyaxial bone screw assembly, the receiver comprising:
a body, comprising:
a rod receiving channel sized and shaped to receive a fixation rod therein;
an axial bore extending longitudinally through the receiver to a distal opening, the distal opening being sized and shaped to receive a head of a bone shank therethrough;
a conical chamber disposed adjacent to the distal opening, the conical chamber being wider at a proximal end of the conical chamber than at a distal end of the conical chamber; and
a ridge protruding from a surface of the axial bore;
a split retainer ring disposed in the conical chamber, the split retainer ring comprising a conical exterior surface, wherein the split retainer ring is configured to expand within the conical chamber to allow a head portion of a bone shank to pass therethrough and thereafter support the head portion in a pivotable relationship; and
a pressure insert disposed at least partly above the split retainer ring, the pressure insert comprising:
a saddle surface configured to seat the fixation rod;
a distally-facing concave surface configured to abut the head portion of the bone shank;
a groove formed in an exterior surface of the pressure insert; and
a rounded corner adjacent to a proximal end of the pressure insert,
wherein the groove is configured to engage the ridge in the receiver to maintain the pressure insert in a first state associated with a first longitudinal position within the receiver;
wherein the pressure insert is configured to flex inward to disengage the groove from the ridge; and
wherein the groove is configured to abut the rounded corner of the pressure insert in a second state associated with a second longitudinal position within the receiver, wherein, in the second state, the groove urges the pressure insert distally such that:
the head portion of the bone shank forms a first friction fit with the concave surface of the pressure insert;
the head portion of the bone shank forms a second friction fit with an interior surface of the split retainer ring; and
the conical exterior surface of the split retainer ring is urged against a surface of the conical chamber.

10. The receiver of claim 9, wherein the split retainer ring comprises an interior concave surface.

11. The receiver of claim 10, wherein the interior concave surface comprises a spherical surface.

12. The receiver of claim 10, wherein:
the receiver comprises an interior conical surface defining the chamber; and
the split retainer ring further comprises an outer conical surface abutting the interior conical surface of the receiver.

13. The receiver of claim 12, wherein the split retainer ring comprises a groove formed in a proximal end of the split retainer ring, and wherein a distal end of the pressure insert is positioned adjacent the groove when the pressure insert is in the second longitudinal position.

14. The receiver of claim 9, wherein the groove extends circumferentially across the exterior surface of the pressure insert.

15. The receiver of claim 14, wherein the groove comprises a non-symmetrical cross-sectional profile.

16. The receiver of claim 9, wherein the ridge comprises a symmetrical cross-sectional profile.

17. A receiver body for a polyaxial bone screw assembly, the receiver body comprising:
a longitudinal axis;
a rod receiving channel oriented transverse to the longitudinal axis and being sized and shaped to receive a fixation rod therein;
an axial bore extending along the longitudinal axis from a proximal opening to a distal opening, the distal opening being sized and shaped to receive a head of a bone shank therethrough;
a conical chamber disposed adjacent to the distal opening and in communication with the axial bore, the conical chamber being wider at a proximal end of the conical chamber than at a distal end of the conical chamber, wherein the conical chamber is wider than the axial bore at both the proximal end and the distal end of the conical chamber;
a ridge protruding inward into the axial bore from an interior surface surrounding the axial bore, the ridge disposed proximally of the conical chamber; and
a set of threads on a proximal portion of the interior surface and disposed proximally of the ridge.

18. The receiver body of claim 17, further comprising a ledge disposed between the conical chamber and the distal opening.

19. The receiver body of claim 17, wherein the ridge comprises a symmetrical cross-sectional profile.

20. The receiver body of claim 19, wherein the symmetrical cross-sectional profile comprises a circular arc, a gaussian curve, or an elliptical arc.

21-22. (canceled)

23. A fastener kit for a spinal fixation system, comprising:
a bone shank comprising a head portion and a distal threaded portion configured to be implanted into bone;
a receiver comprising:
a channel configured to receive a fixation rod therein;
an axial bore extending longitudinally through the receiver from a proximal opening of the receiver to a distal opening, the distal opening being sized and shaped to receive the head portion of the bone shank therethrough;
a chamber disposed adjacent to the distal opening, the chamber comprising a conical interior surface, the conical interior surface comprising a distal portion having a first diameter, and a proximal portion having a second diameter larger than the first diameter; and
a ridge protruding from a surface of the axial bore;
a split retainer ring sized and shaped to be positioned in the chamber; and
a pressure insert sized and shaped to be positioned at least partly above the split retainer ring, the pressure insert comprising:
a saddle configured to seat the fixation rod;
a distally-facing concave surface configured to contact the head portion of the bone shank;
a depression formed in an exterior surface of the pressure insert; and
a rounded corner adjacent to a proximal end of the pressure insert.

24-26. (canceled)

27. A fastener assembly for a spinal fixation system, comprising:
a bone shank comprising a head portion and a distal threaded portion configured to be implanted into bone;
a receiver comprising:
a channel for receiving a fixation rod therein;
an axial bore extending longitudinally through the receiver from a proximal opening of the receiver to a distal opening, the distal opening being sized and shaped to receive the head portion of the bone shank therethrough;
a chamber disposed adjacent to the distal opening, the chamber comprising a conical interior surface, the conical interior surface comprising a distal portion having a first diameter, and a proximal portion having a second diameter larger than the first diameter; and
a depression in the surface of the axial bore;
a split retainer ring disposed in the chamber, the split retainer ring being configured to expand within the chamber to allow the head portion of the bone shank to pass therethrough and thereafter support the head portion in a pivotable relationship; and
a pressure insert disposed at least partly above the split retainer ring, the pressure insert comprising:
a saddle configured to seat the fixation rod;
a distally-facing concave surface configured to contact the head portion of the bone shank; and
a ridge protruding from the exterior surface of the pressure insert;
wherein, when the pressure insert is in a first longitudinal position relative to the receiver, the ridge is configured to engage the depression in the receiver to maintain the pressure insert at the first longitudinal position; and
wherein, when the pressure insert is in a second longitudinal position, the ridge is disengaged from the depression and positioned in a manner that the head portion of the bone shank is maintained in a friction fit with both the pressure insert and the split retainer ring.

28. The fastener assembly of claim 27, wherein the receiver comprises a rounded corner adjacent the depression, and when the pressure insert is in the second longitudinal position, the ridge is positioned adjacent the rounded corner to maintain the bone shank in the friction fit with both the pressure insert and the split retainer ring.

29. The fastener assembly of claim 27. wherein the head portion of the bone shank comprises a spherical surface, wherein the split retainer ring comprises an interior concave surface configured to contact the spherical surface to thereby provide the pivotable relationship.
